(19) United States
(12) Patent Application Publication
(10) Pub. No.: US 2004/0009502 A1
(43) Pub. Date: Jan. 15, 2004

(54) Identification and tissue distribution of two novel spliced variants of the mouse LATS2 gene

(76) Inventors: J.H. David Wu, Pittsford, NY (US); Yi-Guang Chen, Bar Harbor, ME (US); Athanassios Mantalaris, Harrow (GB)

Correspondence Address: Michael L. Goldman, Esq., NIXON PEABODY LLP, Clinton Square, P.O. Box 31051, Rochester, NY, 14603-1051, US

(21) Appl. No.: 10/379,837

(22) Filed: Mar. 4, 2003

Related U.S. Application Data
(60) Provisional application No. 60/361,488, filed on Mar. 4, 2002.

(51) Int. Cl. C12Q 1/68; C07H 21/04; C12N 9/10; C12P 21/02; C12N 1/21

(52) U.S. Cl. 435/6; 435/69.1; 435/193; 435/320.1; 435/325; 536/23.2

(57) ABSTRACT

The present invention relates to isolated nucleic acid molecules encoding splice variants LATS2b and LATS2c, isolated LATS2b and LATS2c protein or polypeptides, and antibodies to the LATS2b and LATS2c proteins or polypeptides. The present invention also relates to methods of using the LATS2b and LATS2c nucleic acid molecules and proteins or polypeptides, including for detecting the expression of LATS2b or LATS2c in a biological sample, regulating LATS2b or LATS2c expression, screening drugs that regulate LATS2b or LATS2c activity and expression, regulating cell growth and differentiation, and treating disease conditions in a subject. Also disclosed are expression vectors, host cells, and transgenic animals transformed with LATS2b and LATS2c nucleic acid molecules.

This application claims the benefit of U.S. Provisional Patent Application Serial No. 60/361,488, filed Mar. 4, 2002.

The subject matter of this application was made with support from the United States Government under National Science Foundation Grant No. BES-9631670 and National Aeronautics and Space Administration Grant No. NAG 8-1382. The U.S. Government may have certain rights.

### FIELD OF THE INVENTION

The present invention relates to nucleic acid molecules encoding spliced versions of mLATS2 and uses thereof.

### BACKGROUND OF THE INVENTION

Rhythmically expressed genes have been reported in a variety of organisms (Green, C. B., “How Cells Tell Time,” Trends in Cell Biology 8(6):224-30 (1998)). In the past few years, differential display techniques have been successfully applied to identify clock-controlled genes (CCGs). For example, differential display-polymerase chain reaction (DD-PCR) was used to identify a gene encoding the protein nocturnin in Xenopus laevis retina (Green et al., “Identification of Vertebrate Circadian Clock-Regulated Genes by Differential Display,” Methods in Molecular Biology 85:219-30 (1997)) and vrille, a transcription factor essential for embryonic development, in Drosophila (Blau et al., “Cycling Vrille Expression is Required for a Functional Drosophila Clock,” Cell 99(6):661-71 (1999)). Subtractive hybridization techniques were used to clone Crg-1, a putative transcription factor regulated by the Drosophila circadian clock (Rouyer et al., “A New Gene Encoding a Putative Transcription Factor Regulated by the Drosophila Circadian Clock,” EMBO J 16(13):3944-54 (1997)), and serotonin N-acetyltransferase (NAT) in pineal gland, a rate-limiting enzyme in melatonin synthesis (Borjigin et al., “Diurnal Variation in mRNA Encoding Serotonin N-acetyltransferase in Pineal Gland,” Nature 378(6559):783-5 (1995)). The ADDER (amplification of double-stranded cDNA end restriction fragment) technique was used to identify clock-controlled genes in the liver (Kornmann et al., “Analysis of Circadian Liver Gene Expression by ADDER, a Highly Sensitive Method for the Display of Differentially Expressed mRNAs,” Nucleic Acids Res 29(11):E51-1 (2001)). More recently, the DNA microarray technique was applied to monitor gene expression levels at different times of the day in cultured fibroblasts (Grundschober et al., “Circadian Regulation of Diverse Gene Products Revealed by mRNA Expression Profiling of Synchronized Fibroblasts,” J Biol Chem 276(50):46751-46758 (2001)) and the Drosophila head (Claridge-Chang et al., “Circadian Regulation of Gene Expression Systems in the Drosophila Head,” Neuron 32(4):657-71 (2001)). These studies have shed tremendous insights on the output pathways of the circadian clock. In particular, it has been shown that transcription factor DBP is a clock-controlled gene directly regulated by CLOCK and BMAL1 heterodimers in the liver (Ripperger et al., “CLOCK, an Essential Pacemaker Component, Controls Expression of the Circadian Transcription Factor DBP,” Genes & Development 14(6):679-89 (2000)). Rhythmic accumulation of DBP then drives circadian expression of its target genes including steroid 15 alpha-hydroxylase (Cyp2a4) and coumarin 7-hydroxylase (Cyp2a5) (Lavery et al., “Circadian Expression of the Steroid 15 Alpha-hydroxylase (Cyp2a4) and Coumarin 7-hydroxylase (Cyp2a5) Genes in Mouse Liver is Regulated by the PAR Leucine Zipper Transcription Factor DBP,” Molecular & Cellular Biology 19(10):6488-99 (1999)). Therefore, an output pathway from the clock system to rhythmic expression of metabolic enzymes through the transcription factor DBP is clearly demonstrated.

The expression and oscillation of mPer1 and mPer2 in murine bone marrow and immediate induction of mPer1 by dexamethasone and phorbol-12-myristate-13-acetate (PMA) in the freshly isolated bone marrow subpopulations has been described (Balsalobre et al., “Multiple Signaling Pathways Elicit Circadian Gene Expression in Cultured Rat-1 Fibroblasts,” Curr. Biol. 10, 1291-1294 (2000); Balsalobre et al., “Resetting of Circadian Time in Peripheral Tissues by Glucocorticoid Signaling,” Science 289:2344-2347 (2000)), and the data strongly suggest the existence of a functional clock system in bone marrow. However, whether hematopoiesis is under the control of the bone marrow clock remains to be determined. It is possible that the clock system in bone marrow receives signals from the central clock and coordinates expression of locally regulated clock-controlled genes that in turn modulate hematopoiesis. To address this question, a direct strategy is to show that the activities and/or expression levels of hematopoietic regulators are controlled by the bone marrow circadian clock. Therefore, identification of cycling gene expression in bone marrow seems to be a straightforward approach to link the circadian clock in bone marrow and hematopoiesis together, although it is possible that post-transcriptional modifications also play roles in adjusting the functions of hematopoietic regulators.

The warts/lats (large tumor suppressor gene) gene was first identified in Drosophila as a tumor suppressor gene (Xu et al., “Identifying Tumor Suppressors in Genetic Mosaics: the Drosophila Lats Gene Encodes a Putative Protein Kinase,” Development 121(4):1053-63 (1995)). The human and mouse homologues of the warts/lats gene, lats1 and lats2, have been recently identified (Tao et al., “Human Homologue of the Drosophila Melanogaster Lats Tumour Suppressor Modulates CDC2 Activity,” Nature Genetics 21(2):177-81 (1999); Nishiyama et al., “A Human Homolog of Drosophila Warts Tumor Suppressor, h-warts, Localized to Mitotic Apparatus and specifically Phosphorylated During Mitosis,” FEBS Letters 459(2):159-65 (1999); Yabuta et al., “Structure, Expression, and Chromosome Mapping of LATS2, a Mammalian Homologue of the Drosophila Tumor Suppressor Gene Lats/Warts,” Genomics 63(2):263-70 (2000); Hori et al., “Molecular Cloning of a Novel Human Protein Kinase, kpm, That is Homologous to Warts/Lats, a Drosophila Tumor Suppressor,” Oncogene 19:3101-3109 (2000). The lats gene encodes a serine/threonine kinase domain highly homologous to the catalytic domain of the myotonic dystrophy protein kinase (DMPK) family. The DMPK family proteins such as Dbf2 and Orb6 in yeast and Citron-K kinase in human have been shown to be involved in mitosis. The kinase activity of Dbf2 is cell-cycle-regulated with its activity peaking in the late mitotic phase (Toyn et al., “The Dbf2 and Dbf20 Protein Kinases of Budding Yeast are Activated After the Metaphase to Anaphase Cell Cycle transition,” EMBO J. 13(5):1103-13 (1994)). For the temperature-sensitive Dbf2 mutant under the non-permissive temperature, the cells arrest in telophase with elongated spindles. Orb6 is required to maintain polarity of the actin cytoskeleton during interphase and to promote actin reorganization both after mitosis and during activation of bipolar growth (Verde et al., “Fission Yeast orb6, a ser/thr Protein Kinase Related to Mammalian Rho Kinase and Myotonic Dystrophy kinase, is Required for Maintenance of Cell Polarity and Coordinates Cell Morphogenesis With the Cell Cycle,” Proc. Natl. Acad. Sci. USA 95(13):7526-31 (1998)). Overexpression of orb6 leads to an increase in cell length at division, indicating that onset of mitosis was delayed. Citron-K kinase has been shown to localize to the cleavage furrow of dividing cells and overexpression of citron-K kinase results in multinucleated cells (Madaule et al., “Role of Citron kinase as a Target of the Small GTPase Rho in Cytokinesis,” Nature 394(6692):491-4 (1998)).

Evidence indicating the involvement of LATS1 and LATS2 in cell cycle regulation has also evolved. For example, it has been shown that phosphorylation of human LATS1 (hLATS1) is cell-cycle-dependent and the phosphorylated hLATS1 negatively regulates the CDC2 activity by forming the hLATS1-CDC2 complex in the mitotic phase (Tao et al., “Human Homologue of the Drosophila Melanogaster Lats Tumour Suppressor Modulates CDC2 Activity,” Nature Genetics 21(2): 177-81 (1999)). In addition, hLATS1 has been reported to localize at the centrosome in the interphase and translocate towards mitotic spindles in the metaphase and anaphase (Nishiyama et al., “A Human Homolog of Drosophila Warts Tumor Suppressor, h-warts, Localized to Mitotic Apparatus and specifically Phosphorylated During Mitosis,” FEBS Letters 459(2):159-65 (1999)). High incidence of soft-tissue sarcomas and ovarian stromal cell tumors in lats1^{-/-} mice also supports the role of LATS1 in cell-cycle control (St. John et al., “Mice Deficient of Lats1 Develop Soft-Tissue Sarcomas, Ovarian Tumours and Pituitary Dysfunction,” Nature Genetics 21(2):182-6 (1999)). Furthermore, the expression of hLATS2 is induced by p53, a tumor suppressor gene involved in cell-cycle control (Kostic et al., “Isolation and Characterization of Sixteen Novel p53 Response Genes,” Oncogene 19(35):3978-87 (2000)). Finally, the human KPM protein (identical to hLATS2) has been shown to undergo phosphorylation during the mitotic phase and has been suggested to play a role in the progression of mitosis (Hori et al., “Molecular Cloning of a Novel Human Protein Kinase, KPM, That is Homologous to Warts/Lats, a Drosophila Tumor Suppressor,” Oncogene 19:3101-3109 (2000)).

Tumorigenesis is a complex process involving activation of oncogenes and inactivation of tumor suppressor cells (Bishop, “Molecular Themes in Oncogenesis,” Cell 64: 235-248 (1991)), resulting in the overproliferation of cells. Although it is becoming clear that there is an important relationship among cell cycle genes, circadian rhythms, and tumor suppression, very little is known about how these factors operate or interact at the cellular level. What is needed is the identification and characterization of specific clock controlled cell cycle-regulatory genes that are also involved in tumor suppression, and methods of using those genes to diagnose, treat, and prevent tumorigenesis.

The present invention is directed to overcoming these and other deficiencies in the art.

### SUMMARY OF THE INVENTION

The present invention relates to an isolated nucleic acid molecule encoding a LATS2b protein or polypeptide. This nucleic acid molecule either: 1) has a nucleotide sequence of SEQ ID NO: 1; 2) encodes a protein or polypeptide having an amino acid sequence of SEQ ID NO: 2; 3) has a nucleotide sequence that is at least 55% similar to the nucleotide sequence of SEQ ID NO: 1 by basic BLAST analysis; or 4) has a nucleotide sequence that hybridizes to the nucleotide sequence of SEQ ID NO: 1 under stringent conditions characterized by a hybridization buffer comprising 5×SSC buffer at a temperature of 45° C.

The present invention also relates to an isolated nucleic acid molecule encoding a LATS2c protein or polypeptide. The nucleic acid molecule either: 1) has a nucleotide sequence of SEQ ID NO: 3; 2) encodes a protein or polypeptide having an amino acid sequence of SEQ ID NO: 4; 3) has a nucleotide sequence that is at least 55% similar to the nucleotide sequence of SEQ ID NO: 3 by basic BLAST analysis; or 4) has a nucleotide sequence that hybridizes to the nucleotide sequence of SEQ ID NO: 3 under stringent conditions characterized by a hybridization buffer comprising 5×SSC buffer at a temperature of 45° C.

Another aspect of the present invention is an isolated LATS2b protein or polypeptide.

Yet another aspect of the present invention is an isolated antibody which recognizes the LATS2b protein or polypeptide of the invention.

Another aspect of the present invention is a composition having a pharmaceutical carrier and an antibody against an antigen, where the antigen is the isolated LATS2b protein or polypeptide of the invention.

Another aspect of the present invention is an isolated LATS2c protein or polypeptide.

Yet another aspect of the present invention is an isolated antibody which recognizes the LATS2c protein or polypeptide of the invention.

Another aspect of the present invention is a composition having a pharmaceutical carrier and an antibody against an antigen, where the antigen is the isolated LATS2c protein or polypeptide of the invention.

The present invention also relates to a method of detecting the expression of LATS2b in a biological sample. This method involves providing an antibody or binding portion thereof that recognizes the LATS2b polypeptide or protein, contacting the antibody or binding portion thereof with a biological sample, and detecting any binding that occurs between the biological sample and the antibody or binding portion thereof, thereby detecting the expression of LATS2b in the biological sample.

Another aspect of the present invention is a second method of detecting LATS2b expression in a biological sample. This method involves providing a nucleic acid molecule that specifically hybridizes to a gene encoding a LATS2b polypeptide or protein, a probe thereto or primers derived therefrom, contacting the nucleic acid molecule encoding a LATS2b polypeptide or protein, a probe thereto or primers derived therefrom with a biological sample, and detecting whether the nucleic acid molecule has undergone any hybridization, thereby detecting LATS2b expression in the biological sample.

The present invention also relates to a method of treating a disease condition in a subject. This method involves providing a therapeutic amount of a pharmaceutical conjugate having an antibody against a LATS2b protein or polypeptide and a cytotoxic component, and administering the conjugate to a subject under conditions effective to form an immune complex with a LATS2b polypeptide or protein, thereby treating a disease condition.

The present invention also relates to a method of detecting the expression of LATS2c in a biological sample. This method involves providing an antibody or binding portion thereof which recognizes the LATS2c polypeptide or protein, contacting the antibody or binding portion thereof with a biological sample, and detecting any binding that occurs between the biological sample and the antibody or binding portion, thus detecting the expression of LATS2c in the biological sample.

Another aspect of the present invention is a second method of detecting LATS2c expression in a biological sample. This method involves providing a nucleic acid molecule that specifically hybridizes to a gene encoding a LATS2c polypeptide or protein, a probe thereto or primers derived therefrom, contacting the nucleic acid molecule encoding a LATS2c polypeptide or protein, a probe thereto or primers derived therefrom with the biological sample, and detecting whether the nucleic acid molecule has undergone any hybridization, thus detecting LATS2c expression in the biological sample.

Another aspect of the present invention is a method of treating a disease condition in a subject. This method involves providing a therapeutic amount of a pharmaceutical conjugate having an antibody against a LATS2c protein or polypeptide and a cytotoxic component, and administering the conjugate to a subject under conditions effective to form an immune complex with a LATS2c polypeptide or protein, thereby treating a disease condition in the subject.

Another aspect of the present invention is a method of regulating LATS2b expression in a subject. This method involves administering to the subject an antisense nucleic acid, which is complementary to the nucleic acid molecule that either: 1) has a nucleotide sequence of SEQ ID NO: 1; 2) encodes a protein or polypeptide having an amino acid sequence of SEQ ID NO: 2; 3) has a nucleotide sequence that is at least 55% similar to the nucleotide sequence of SEQ ID NO: 1 by basic BLAST analysis; or 4) has a nucleotide sequence that hybridizes to the nucleotide sequence of SEQ ID NO: 1 under stringent conditions characterized by a hybridization buffer comprising 5×SSC buffer at a temperature of 45° C., thereby regulating LATS2b expression in the subject.

The present invention also relates to a method of regulating LATS2c expression in a subject. This method involves administering to the subject the antisense nucleic acid molecule which is complementary to the nucleic acid molecule that either: 1) has a nucleotide sequence of SEQ ID NO: 3; 2) encodes a protein or polypeptide having an amino acid sequence of SEQ ID NO: 4; 3) has a nucleotide sequence that is at least 55% similar to the nucleotide sequence of SEQ ID NO: 3 by basic BLAST analysis; or 4) has a nucleotide sequence that hybridizes to the nucleotide sequence of SEQ ID NO: 3 under stringent conditions characterized by a hybridization buffer comprising 5×SSC buffer at a temperature of 45° C.

Another aspect of the present invention is a method of gene therapy that involves administering to a subject the nucleic acid molecule encoding a LATS2b protein or polypeptide a fragment thereof, or a vector expressing a LATS2b protein, polypeptide or fragment thereof.

The present invention also relates to another method of gene therapy. This method involves administering to a subject the nucleic acid molecule encoding a LATS2c protein or polypeptide, a fragment thereof, or a vector expressing LATS2c protein, polypeptide or fragment thereof.

Another aspect of the present invention is a transgenic animal having an altered expression of LATS2b.

The present invention also relates to a transgenic animal whose somatic and germ cells lack a gene encoding a LATS2b protein or polypeptide, or possess a disruption in that gene, whereby the animal exhibits a lack of expression of LATS2b.

Another aspect of the present invention is a transgenic animal having an altered expression of LATS2c.

Another aspect of the present invention is a transgenic animal whose somatic and germ cells lack a gene encoding a LATS2c protein or polypeptide, or possess a disruption in that gene, whereby the animal exhibits a lack of expression of LATS2c.

The present invention also relates to a method of screening drugs that regulate LATS2b activity. This method involves providing an isolated LATS2b protein or polypeptide, a reagent upon which LATS2b exerts activity, and a test compound. The LATS2b protein or polypeptide, the reagent, and the test compound are blended to form a mixture. The activity of LATS2b upon the reagent in the mixture is determined, and any difference between the activity of LATS2b upon the reagent with and without the test compound is measured.

Another aspect of the present invention relates to a method of screening for drugs that regulate LATS2c activity. This method involves providing the isolated LATS2c protein or polypeptide of the invention, a reagent upon which LATS2c exerts activity, and a test compound. The LATS2c protein or polypeptide, the reagent, and the test compound are blended to form a mixture. The activity of LATS2c upon the reagent in the mixture is determined, and any difference between the activity of LATS2c upon the reagent with and without the test compound is measured.

The present invention also relates to a method of screening for drugs that regulate LATS2b expression. This method involves transforming a host cell with a nucleic acid construct having a nucleic acid molecule encoding a LATS2b protein or polypeptide operably linked to transcriptional and translational regulatory elements, culturing the transformed cells, adding a test compound to the culture containing the transformed cells, and determining whether the test compound regulates the expression of LATS2b in the transformed cells.

Another aspect of the present invention is a method of screening for drugs that regulate LATS2b expression. This method involves isolating cells from a transgenic animal having an altered expression of LATS2b, adding a test compound to the isolated cells, and determining whether the test compound regulates the expression of LATS2b in the isolated cells.

The present invention also relates to a method of screening for drugs that regulate LATS2c expression. This method involves transforming a host cell with a nucleic acid construct having a nucleic acid molecule encoding a LATS2c protein or polypeptide operably linked to transcriptional and translational regulatory elements, culturing the transformed cells, adding a test compound to the culture containing the transformed cells, and determining whether the test compound regulates the expression of LATS2c in the transformed cells.

The present invention also relates to another method of screening for drugs that regulate LATS2c expression. This method involves isolating cells from a transgenic animal having an altered expression of LATS2c, adding a test compound to the isolated cells, and determining whether the test compound regulates the expression of LATS2c in the cells.

Another aspect of the present invention is a method of treating a disease condition in a subject. This method involves providing a nucleic acid molecule encoding a LATS2b protein or polypeptide or probe thereto, and contacting the nucleic acid molecule encoding a LATS2b protein or polypeptide or probe thereto with a cell or tissue sample of a subject under conditions effective to bind to cells overexpressing LATS2b from the cell or tissue sample, and removing cells or tissues which are selected by the nucleic acid molecule or probe thereto, thereby treating a disease condition in the subject.

The present invention also relates to another method of treating a disease condition in a subject. This method involves providing a labeled antibody or binding protein thereof, that recognizes the LATS2b protein or polypeptide or a fragment thereof contacting the antibody or binding protein thereof that recognizes the LATS2b protein or polypeptide or a fragment thereof with a cell or tissue sample of the subject under conditions effective to bind to cells overexpressing LATS2b from the cell or tissue sample, and removing cells or tissues which are selected by the antibody or binding protein thereof, thereby treating a disease condition.

Another aspect of the present invention is another method of treating a disease condition in a subject. This method involves providing a nucleic acid molecule encoding a LATS2c protein or polypeptide or a probe thereto, contacting the nucleic acid molecule encoding the LATS2c protein or polypeptide or the probe thereto with a cell or tissue sample of the subject under conditions effective to bind to cells overexpressing LATS2c from the cell or tissue sample, and removing the cells or tissues which are selected by the nucleic acid molecule or probe thereto, thereby treating a disease condition in the subject.

Another aspect of the present invention is another method of treating a disease condition in a subject. This method involves providing a labeled antibody or binding protein thereof that recognizes the LATS2c protein or polypeptide or a fragment thereof; contacting the antibody or the binding protein thereof that recognizes the LATS2c protein or polypeptide or a fragment thereof with a cell or tissue sample of the subject under conditions effective to bind to cells overexpressing LATS2c from the cell or tissue sample, and removing the cells or tissues which are selected by the antibody or binding protein thereof, thereby treating a disease condition in the subject.

The present invention also relates to a vaccine having an antigen including a LATS2b protein or polypeptide or an antigenic fragment thereof and a carrier.

The present invention also relates to yet another method of treating a disease condition in a subject. This method involves administering to a subject a vaccine having an antigen including a LATS2b protein or polypeptide or an antigenic fragment thereof and a carrier.

The present invention also relates to a vaccine including an antigen having a LATS2c protein or polypeptide or an antigenic fragment thereof and a carrier.

Another aspect of the present invention is a method of treating a disease condition in a subject. This method involves administering the vaccine including an antigen having a LATS2c protein or polypeptide or an antigenic fragment thereof, and a carrier, to a subject.

The present invention also relates to a method of regulating cell growth or differentiation. This method involves introducing to cells a vector expressing a LATS2b nucleic acid molecule, thereby regulating the growth or differentiation of the cells.

The present invention also relates to another method of regulating cell growth or differentiation. This method involves introducing to cells a vector expressing a LATS2c nucleic acid molecule, thereby regulating the growth or differentiation of the cells.

Another aspect of the present invention is a method of altering the expression of LATS2 in a cell or subject. This method involves treating a cell with a chemical or molecule capable of interfering with circadian control of the cell, thereby altering the expression of LATS2 in the cell or subject.

Another aspect of the present invention is a method of altering the expression of LATS2b in a cell or subject. This method involves treating a cell with a chemical or molecule capable of interfering with circadian control of the cell, thereby altering the expression of LATS2b in the cell or subject.

Another aspect of the present invention is a method of altering the expression of LATS2c in a cell or subject. This method involves treating a cell or subject with a chemical or molecule capable of interfering with circadian control of the cell, thereby altering the expression of LATS2c in the cell or subject.

### BRIEF DESCRIPTION OF THE DRAWINGS

FIGS. 1A-B are the nucleotide and deduced amino acid sequences of the genes encoding mLATS2b and mLATS2c. FIG. 1A is the nucleotide sequence (SEQ ID NO: 1) and the amino acid sequence (SEQ ID NO: 2) of mLATS2b, from clone 3-1. FIG. 1B is the nucleotide sequence (SEQ ID NO: 3) and the amino acid sequence (SEQ ID NO: 4) of mLATS2c, from clone 3-3. The 3′-RACE products were obtained using Forward Primers 1 or 2, as indicated by long arrows on the top. The stop codon is indicated by an asterisk. The start codon is assigned according to the mLATS2 sequence (GenBank accession number BAA92380, which is hereby incorporated by reference in its entirety). The putative splicing site is indicated by a short arrow. The putative polyadenylation signal is boxed. The numbers denote the positions of the first nucleotides or last amino acids of each line.

FIGS. 2A-B are the expression pattern of a differentially displayed band (6A-2-9), confirmed by relative quantitative RT-PCR. FIG. 2A is a comparison of gene expression patterns at six circadian times. The position of band 6A-2-9 is indicated by an arrowhead. In FIG. 2B, relative quantitative RT-PCR confirms the expression pattern of band 6A-2-9. *p<0.05 as compared to the values at 4, 8, and 16 hours after light onset (t test). The intensity of the DNA band corresponding to mlats2 or mlats2b was normalized to that of the 18S rRNA internal control. Within each experiment, the highest normalized level was set as 100 and the relative amounts of mRNA at other time points were calculated. Each value represents the mean ±SEM from the results of three mice. The horizontal bar at the bottom represents the light-dark cycle. Data at 0 and 20 hours after light onset are plotted twice.

FIG. 3 is a schematic diagram comparing mLATS2, mLATS2b, and mLATS2c. The numbers denote the positions of amino acids. The N-terminal 113 amino acids (black box) are identical for all three proteins. The insertion of 49 amino acids in mLATS2c is shown by an open box. The meshed box indicates the identical region between mLATS2b and mLATS2c. (The figure is not drawn to scale.)

FIG. 4 is an agarose gel showing two cDNA fragments, designated mlats2b and mlats2c, (indicated by arrows) amplified by 3′-RACE using murine bone marrow cDNA as the template and Forward Primer 1 as the gene-specific primer. M: DNA size markers.

FIG. 5 is a genomic Southern blot analysis of the mouse lats2 gene. Mouse genomic DNA was digested by Pst I and separated on a 0.8% agarose gel. DNA was transferred to a positively charged nylon membrane and hybridized with a probe within the region common to mlats2, mlats2b, and mlats2c (nucleotides 67 to 389 in mlats2b in FIG. 1A). A single band of about 1.6 kb is indicated by an arrow.

FIG. 6 is a gel electrophoresis of RT-PCR performed in the presence (+) or absence (−) of reverse transcriptase to analyze the expression of mlats2, mlats2b, and mlats2c in murine bone marrow. The primer sets were designed to specifically amplify each of the spliced variants. The PCR products of mlats2 (483 bp), mlats2b (379 bp), and mlats2c (525 bp) are indicated by white arrowheads.

FIGS. 7A-B show the expression of mlats2, mlats2b, and mlats2c in different mouse tissues. FIG. 7A is an agarose gel electrophoresis showing expression of mlats2, mlats2b, and mlats2c in various tissues determined by RT-PCR. FIG. 7B shows the relative expression levels of mlats2, mlats2b, and mlats2c in various murine tissues. The amounts of the three lats2 transcripts were normalized to the β-actin signal. The normalized level of mlats2 in testis was set as 100. The ratio of mlats2b/mlats2 or mlats2c/mlats2 in each tissue is indicated.

FIG. 8 is a partial sequence alignment of mlats2b/mlats2c (SEQ ID NO: 5), mlats2 (SEQ ID NO: 6), hlats2/kpm (SEQ ID NO: 7), and the corresponding human genomic DNA (chromosome 13)(SEQ ID NO: 8, line starting position 185393; SEQ ID NO: 9, line starting position 185333; SEQ ID NO: 10, line starting position 135674; SEQ ID NO: 11, line starting 135709) near the putative splicing site. The GenBank accession numbers are AF207547 (hlats2/kpm), AB023958 (mlats2), and AL161613 (human genomic DNA). The arrowhead represents the putative splicing site shown in FIG. 1A. The sequences at both ends of the putative intron sequence are underlined and the consensus 5′-splice donor (GT) and the 3′-splice acceptor (AG) are capitalized. Omitted sequence in human genomic DNA is indicated as dashes. The human genomic DNA sequence shown here is derived from two unordered and non-overlapping fragments in AL161613. The complete sequence of the intron remains to be determined. Identical nucleotides are indicated by asterisks.

FIG. 9 is a comparison of mLATS2 (SEQ ID NO: 12) and hLATS2/KPM (SEQ ID NO: 13). The top panel is a schematic diagram showing that the N-terminal region and the kinase domains are highly conserved. The percentages of identity are indicated in between the sequences. The horizontal bar indicates the approximate size of 100 amino acids. The bottom panel shows the sequence alignment of the N-terminal regions. The GenBank accession numbers are BAA92380 (mLATS2) and AAF80561 (hLATS2/KPM). Identical residuals are shown by shaded background. The gap is indicated by a dash.

FIGS. 10A-B are circadian expression profiles of mlats2 and mlats2b in total bone marrow cells. FIG. 10A shows the relative amount of mlats2 mRNA at different times. *p<0.05 as compared to the values at 4 hours after light onset (t test). FIG. 10B shows the relative amount of mlats2b mRNA at different times. *p<0.05 as compared to the values at 4 and 20 hours after light onset (t test). The intensity of the DNA band corresponding to mlats2 or mlats2b was normalized to that of the 18S rRNA internal control. Within each experiment, the highest normalized level was set as 100 and the relative amounts of mRNA at other time points were calculated. Each value represents the mean ±SEM of the results from three mice. The horizontal bar at the bottom represents the light-dark cycle. Data at 0 and 20 hours after light onset are plotted twice.

FIGS. 11A-B are schematic diagrams showing the positions of the inserts used in the yeast two-hybrid and mammalian one-hybrid assays. The numbers on top of the bars denote the positions of the amino acids. mLATS2N373 and mLATS2N96 refer to the truncated forms of the mLTATS2 gene, encoding the N-terminal 373 and 96 amino acids, respectively. mRBT1N121 and mBBT1C76 refer to the truncated forms of the mRBT1C76 gene, encoding the N-terminal 121 and C-terminal 76 amino acids, respectively.

FIG. 12 shows the mapping of the protein-protein interaction region between mouse Replication Protein Binding Trans-Activator (mRBT1) and mLATS2/2b. Yeast cells (AH109) were transformed with plasmids pGBKT7 and pGADT7 (0.5 μg each) containing indicated inserts. After transformation, cells were plated on double dropout plates (2DO; -Leu/-Trp) and quadruple dropout plates (4DO; -Ade/-His/-Leu/-Trp) to determine successful transformation and the protein-protein interaction respectively. Plates were incubated at 30° C. for 2 (2DO) or 5 (4DO) days.

FIG. 13 shows the interaction-dependent regulation of mRBT1 transcriptional activity by mLATS2. NIH3T3 cells were transfected with indicated GAL4-fusion protein expression plasmids and pcDNA3-mLATS2 (white bars) or the pcDNA3 empty vector (black bars). The luciferase activities in the absence of the mLATS2 expression plasmid were set as 100. Data are presented as mean ±SEM from the results of six samples in two independent experiments.

FIG. 14 is a graph showing that the kinase domain of mLATS2 is essential for its inhibitory effect on mRBT1. NIH3T3 cells were transfected with the GAL4-mRBT1 expression plasmid and indicated amounts (ng) of the mLATS2 or mLATS2N373 expression plasmid. The luciferase activity in the absence of mLATS2 and mLATS2N373 is set as 100. The data are presented as mean ±SEM from the results of six samples in two independent experiments.

FIG. 15 is a graph showing that the inhibitory effect of mLATS2 on mRBT1 transcriptional activity is antagonized by mLATS2b. NIH3T3 cells were transfected with the GAL4-mRBT1 expression plasmid and indicated amounts (ng) of the mLATS2 and mLATS2b expression plasmids. The luciferase activity in the absence of mLATS2 and mLATS2b is set as 100. The data are presented as mean ±SEM from the results of six samples in two independent experiments.

### DETAILED DESCRIPTION OF THE INVENTION

The present invention relates to an isolated nucleic acid molecule encoding a LATS2b protein or polypeptide. This nucleic acid molecule, mlats2b herein, has a nucleotide sequence of SEQ ID NO: 1 as follows:

| | |
|---|---|
| cactgacact gttgactgtt ctctttaaaa taataagacg ctttgagaag attgtattta | 60 |
| tggtaaaagg aaactggact aacaatgagg ccaaagactt ttcctgccac aacttactct | 120 |
| ggaaatagcc ggcagcgatt gcaagagatt cgagaggggc tgaagcagcc atccaaggct | 180 |
| tccacccagg ggctgctggt gggaccaaac agtgacactt ccctggatgc caaagtcctg | 240 |
| gggagcaaag atgcctccag gcagcagcaa atgagagcca ccccgaagtt tggaccttat | 300 |
| caaaaagctc tcagggaaat ccgatattcc ctcctgcctt ttgccaacga gtcaggcact | 360 |
| tcggcagctg cagaggtgaa ccggcagatg cttcaggagt tggtgaatgc gggatgtgac | 420 |
| cagatgcata ttcctggtgc gtgtctgttt ctggagatgc tcctgtctgt ccctcccatc | 480 |
| tcccaaacag cagcacctgg attacaggca cacaggctgc tacagctttg cagtgtgtgc | 540 |
| gaattcaagc tcggaccctc acacttgtac ctgaagcacg gagccagccg tcttctcagc | 600 |
| cccttatgtc cataatacta gggcttgatt ctgaacgtga gaggaaatgt ggcacttggc | 660 |
| tttctgaact tggctgattt tgctccatgg atgacctcaa attgcatcca tggttacagt | 720 |
| tttttgtcat tcttacaaat gtgactttgt ccttcgatat ggcctaataa aacgcctttg | 780 |
| tgcttaaaaa aaaaaaaaaa aaaaaaaa 808 | |

The mlats2b nucleic acid molecule of the present invention, isolated from mouse, encodes a protein or polypeptide, LATS2b herein, having an amino acid sequence of SEQ ID NO: 2 as follows:

```
Met Arg Pro Lys Thr Phe Pro Ala Thr Thr Tyr Ser
    1 5 10
Gly Asn Ser Arg Gln Arg Leu Gln Glu Ile Arg Glu
                  15 20
Gly Leu Lys Gln Pro Ser Lys Ala Ser Thr Gln Gly
  25 30 35
Leu Leu Val Gly Pro Asn Ser Asp Thr Ser Leu Asp
                          40 45
Ala Lys Val Leu Gly Ser Lys Asp Ala Ser Arg Gln
          50 55 60
Gln Gln Met Arg Ala Thr Pro Lys Phe Gly Pro Tyr
                                  65 70
Gln Lys Ala Leu Arg Glu Ile Arg Tyr Ser Leu Leu
                                    75 80
Pro Phe Ala Asn Glu Ser Gly Thr Ser Ala Ala Ala
  85 90 95
Glu Val Asn Arg Gln Met Leu Gln Glu Leu Val Asn
                        100 105
Ala Gly Cys Asp Gln Met His Ile Pro Gly Ala Cys
        110 115 120
Leu Phe Leu Glu Met Leu Leu Ser Val Pro Pro Ile
                                125 130
Ser Gln Thr Ala Ala Pro Gly Leu Gln Ala His Arg
                135 140
Leu Leu Gln Leu Cys Ser Val Cys Glu Phe Lys Leu
145 150 155
Gly Pro Ser His Leu Tyr Leu Lys His Gly Ala Ser
                        160 165
Arg Leu Leu Ser Pro Leu Cys Pro
        170 175
```

The present invention relates to an isolated nucleic acid molecule encoding a LATS2c protein or polypeptide. This nucleic acid molecule, mlats2c herein, has a nucleotide sequence of SEQ ID NO: 3 as follows:

| | |
|---|---|
| cactgacact gttgactgtt ctctttaaaa taataagacg ctttgagaag attgtattta | 60 |
| tggtaaaagg aaactggact aacaatgagg ccaaagactt ttcctgccac aacttactct | 120 |
| ggaaatagcc ggcagcgatt gcaagagatt cgagaggggc tgaagcagcc atccaaggct | 180 |
| tccacccagg ggctgctggt gggaccaaac agtgacactt ccctggatgc caaagtcctg | 240 |
| gggagcaaag atgcctccag gcagcagcaa atgagagcca ccccgaagtt tggaccttat | 300 |
| caaaaagctc tcagggaaat ccgatattcc ctcctgcctt ttgccaacga gtcaggcact | 360 |
| tcggcagctg cagaggtgaa ccggcagatg cttcaggagt tggtgaatgc gggatgtgac | 420 |
| caggtggcct tgaactcaca gagatatgtt tgcctcagcc tctcaagtgc tggggtgaaa | 480 |
| ggcctgtgtc agaaatgcgt cttcataagg aaggtatcag tggctgatcg cctgtgttcc | 540 |
| aggctgtgga agatcttgaa ccggtcaaca atgcatattc ctggtgcgtg tctgtttctg | 600 |
| gagatgctcc tgtctgtccc tcccatctcc caaacagcag cacctggatt acaggcacac | 660 |
| aggctgctac agctttgcag tgtgtgcgaa ttcaagctcg gaccctcaca cttgtacctg | 720 |
| aagcacggag ccagccgtct tctcagcccc ttatgtccat aatactaggg cttgattctg | 780 |
| aacgtgagag gaaatgtggc acttggcttt ctgaacttgg ctgattttgc tccatggatg | 840 |
| acctcaaatt gcatccatgg ttacagtttt ttgtcattct tacaaatgtg actttgtcct | 900 |
| tcgatatggc ctaataaaac gcctttgtgc ttaaaaaaaa aaaaaaaaaa aaaaa 955 | |

The mlats2c nucleic acid molecule of the present invention, isolated from mouse, encodes a protein or polypeptide, LATS2c herein, having an amino acid sequence of SEQ ID NO: 4 as follows:

```
Met Arg Pro Lys Thr Phe Pro Ala Thr Thr Tyr Ser
    1 5 10
Gly Asn Ser Arg Gln Arg Leu Gln Glu Ile Arg Glu
                  15 20
Gly Leu Lys Gln Pro Ser Lys Ala Ser Thr Gln Gly
  25 30 35
Leu Leu Val Gly Pro Asn Ser Asp Thr Ser Leu Asp
                          40 45
Ala Lys Val Leu Gly Ser Lys Asp Ala Ser Arg Gln
          50 55 60
Gln Gln Met Arg Ala Thr Pro Lys Phe Gly Pro Tyr
                                  65 70
Gln Lys Ala Leu Arg Glu Ile Arg Tyr Ser Leu Leu
                  75 80
Pro Phe Ala Asn Glu Ser Gly Thr Ser Ala Ala Ala
  85 90 95
Glu Val Asn Arg Gln Met Leu Gln Glu Leu Val Asn
                        100 105
Ala Gly Cys Asp Gln Val Ala Leu Asn Ser Gln Arg
        110 115 120
Tyr Val Cys Leu Ser Leu Ser Ser Ala Gly Val Lys
                                125 130
Gly Leu Cys Gln Lys Cys Val Phe Ile Arg Lys Val
                135 140
Ser Val Ala Asp Arg Leu Cys Ser Arg Leu Trp Lys
145 150 155
Ile Leu Asn Arg Ser Thr Met His Ile Pro Gly Ala
                        160 165
Cys Leu Phe Leu Glu Met Leu Leu Ser Val Pro Pro
        170 175 180
Ile Ser Gln Thr Ala Ala Pro Gly Leu Gln Ala His
                                185 190
Arg Leu Leu Gln Leu Cys Ser Val Cys Glu Phe Lys
                195 200
Leu Gly Pro Ser His Leu Tyr Leu Lys His Gly Ala
205 210 215
Ser Arg Leu Leu Ser Pro Leu Cys Pro
                        220 225
```

Also suitable as an isolated nucleic acid molecule according to the present invention is a nucleic acid which has a nucleotide sequence that is at least 55% similar to the nucleotide sequence of SEQ ID NO: 1 and/or SEQ ID NO: 3 by basic BLAST using default parameters analysis. Also suitable as an isolated nucleic acid molecule according to the present invention is an isolated nucleic acid molecule encoding a LATS2b and/or LATS2c protein, where the nucleic acid hybridizes to the nucleotide sequence of SEQ ID NO: 1 and/or SEQ ID NO: 3, respectively, under stringent conditions characterized by a hybridization buffer comprising 5×SSC buffer at a temperature of 45° C. For the purposes of defining a suitable level of stringency, reference can conveniently be made to Sambrook et al., Molecular Cloning: A Laboratory Manual, Third Edition, Cold Spring Harbor: Cold Spring Harbor Laboratory Press, New York (2001); Nucleic Acid Hybridization: A Practical Approach, Hames and Higgins, Eds., Oxford:IRL Press (1988); and Hybridization with cDNA Probes User Manual, Clonetech Laboratories, CA (2000), which are hereby incorporated by reference in their entirety). Another example of high stringency conditions is 4-5×SSC/0.1% w/v SDS at 54° C. for 1-3 hours. Another stringent hybridization condition is hybridization at 4×SSC at 65° C., followed by a washing in 0.1×SSC at 65° C. for about one hour. Alternatively, an exemplary stringent hybridization condition is in 50% formamide, 4×SSC, at 42° C. Still another example of stringent conditions include hybridization at 62° C. in 6×SSC, 0.05× BLOTTO, and washing at 2×SSC, 0.1% SDS at 62° C. The skilled artisan is aware of various parameters which may be altered during hybridization and washing and which will either maintain or change the stringency conditions, including temperature, salt, the presence of organic solvents, the size (i.e., number of nucleotides) and the G-C content of the nucleic acids involved, as well as the hybridization assay employed.

Typically, the proteins or polypeptides of the present invention are secreted into the growth medium of recombinant E. coli. To isolate the desired protein, the E. coli host cell carrying a recombinant plasmid is propagated, homogenized, and the homogenate is centrifuged to remove bacterial debris. The supernatant is then subjected to sequential ammonium sulfate precipitation. The fraction containing the desired protein of the present invention is subjected to gel filtration in an appropriately sized dextran or polyacrylamide column to separate the proteins. If necessary, the protein fraction may be further purified by HPLC. Alternative methods may be used as suitable.

Mutations or variants of the above polypeptides or proteins are encompassed by the present invention.

Variants may be modified by, for example, the deletion or addition of amino acids that have minimal influence on the properties, secondary structure, and hydropathic nature of the desired polypeptide. For example, a polypeptide may be conjugated to a signal (or leader) sequence at the N-terminal end of the protein which co-translationally or post-translationally directs transfer of the protein. The polypeptide may also be conjugated to a linker or other sequence for ease of synthesis, purification, or identification of the polypeptide.

Fragments of the above proteins are also encompassed by the present invention. Suitable fragments can be produced by several means. In the first, subclones of the gene encoding the desired protein of the present invention are produced by conventional molecular genetic manipulation by subcloning gene fragments. The subclones then are expressed in vitro or in vivo in bacterial cells to yield a smaller protein or peptide.

In another approach, based on knowledge of the primary structure of the proteins of the present invention, fragments of the genes of the present invention may be synthesized by using the polymerase chain reaction (“PCR”) technique together with specific sets of primers chosen to represent particular portions of the protein. These then would be cloned into an appropriate vector for increased expression of an accessory peptide or protein.

Chemical synthesis can also be used to make suitable fragments. Such a synthesis is carried out using known amino acid sequences for the proteins of the present invention. These fragments can then be separated by conventional procedures (e.g., chromatography, SDS-PAGE) and used in the methods of the present invention.

The LATS2b and LATS2c proteins or polypeptides of the present invention are characterized herein as cell-cycle regulators (see Example 15, below). Accordingly, in one aspect of the present invention the isolated proteins or polypeptides of the present invention have an N-terminus which binds to a cell-cycle related protein. Exemplary cell-cycle related proteins, without limitation, include zyxin and RBT1.

The nucleic acid molecule encoding a LATS2b or LATS2c polypeptide or protein can be introduced into an expression system or vector of choice using conventional recombinant technology. Generally, this involves inserting the nucleic acid molecule into an expression system to which the molecule is heterologous (i.e., not normally present). The heterologous nucleic acid molecule is inserted into the expression system or vector in proper sense (5′→3′) orientation and correct reading frame. Alternatively, the nucleic acid may be inserted in the “antisense” orientation, i.e, in a 3′→5′ prime direction. The vector contains the necessary elements for the transcription and translation of the inserted protein-coding sequences.

Antisense nucleic acids are DNA or RNA molecules or oligoribonucleotides or oligodeoxyribonucleotides that are complementary to at least a portion of a specific mRNA molecule. Weintraub, Scientific American 262:40 (1990), which is hereby incorporated by reference in its entirety. In the cell, the antisense nucleic acids hybridize to a target nucleic acid. The specific hybridization of an antisense nucleic acid molecule with its target nucleic acid interferes with the normal function of the target nucleic acid. The functions of DNA to be interfered with include replication and transcription. The functions of RNA to be interfered with include all vital functions, for example, translocation of the RNA to the site of protein translation, translation of protein from the RNA, splicing of the RNA to yield one or more mRNA species, and catalytic activity which may be engaged in or facilitated by the RNA. The overall effect of such interference with target nucleic acid function is the regulation of the protein expression. In the context of the present invention, “regulation” of expression means either an increase (up-regulation) or a decrease (down-regulation) in the expression of a nucleic acid encoding LATS2b or LATS2c. U.S. Pat. No. 6,204,374 to Sidransky; U.S. Pat. No. 6,335,194 to Bennett et al., which are hereby incorporated by reference in their entirety.

In any aspect of the present invention in which down-regulation of LATS2b or 2c expression is desired, the method may involve an RNA-based form of gene-silencing known as RNA-interference (RNAi). Numerous reports have been published on critical advances in the understanding of the biochemistry and genetics of both gene silencing and RNAi (Matzke et al., “RNA-Based Silencing Strategies in Plants,” Curr. Opin. Genet. Dev. 11(2):221-227 (2001), which is hereby incorporated by reference in its entirety). In RNAi, the introduction of double stranded RNA (dsRNA, or iRNA, for interfering RNA) into animal or plant cells leads to the destruction of the endogenous, homologous mRNA, phenocopying a null mutant for that specific gene. In both post-transcriptional gene silencing and RNAi, the dsRNA is processed to short interfering molecules of 21-, 22- or 23-nucleotide RNAs (siRNA) by a putative RNAaseIII-like enzyme (Tuschl T., “RNA Interference and Small Interfering RNAs,” Chembiochem 2: 239-245 (2001); Zamore et al., “RNAi: Double Stranded RNA Directs the ATP-Dependent Cleavage of mRNA at 21 to 23 Nucleotide Intervals,” Cell 101, 25-3, (2000), which are hereby incorporated by reference in their entirety). The endogenously generated siRNAs mediate and direct the specific degradation of the target mRNA. In the case of RNAi, the cleavage site in the mRNA molecule targeted for degradation is located near the center of the region covered by the siRNA (Elbashir et al., “RNA Interference is Mediated by 21 - and 22-Nucleotide RNAs,” Gene Dev. 15(2):188-200 (2001), which is hereby incorporated by reference in its entirety). In one aspect, dsRNA for the nucleic acid molecule of the present invention can be generated by transcription in vivo. This involves modifying the nucleic acid molecule of the present invention for the production of dsRNA, inserting the modified nucleic acid molecule into a suitable expression vector having the appropriate 5′ and 3′ regulatory nucleotide sequences operably linked for transcription and translation, and introducing the expression vector having the modified nucleic acid molecule into a suitable host cell or subject. In another aspect of the present invention, complementary sense and antisense RNAs derived from a substantial portion of the coding region of the nucleic acid molecule of the present invention are synthesized in vitro. (Fire et al., “Specific Interference by Ingested dsRNA,” Nature 391:806-811 (1998); Montgomery et al, “RNA as a Target of Double-Stranded RNA-Mediated Genetic Interference in Caenorhabditis elegans,” Proc. Natl Acad Sci USA 95: 15502-15507; Tabara et al., “RNAi in C. elegans: Soaking in the Genome Sequence,” Science 282:430-431 (1998), which are hereby incorporated by reference in their entirety). The resulting sense and antisense RNAs are annealed in an injection buffer, and dsRNA is administered to the subject using any method of administration described herein, infra.

U.S. Pat. No. 4,237,224 to Cohen and Boyer, which is hereby incorporated by reference in its entirety, describes the production of expression systems in the form of recombinant plasmids using restriction enzyme cleavage and ligation with DNA ligase. These recombinant plasmids are then introduced by means of transformation and replicated in unicellular cultures including prokaryotic organisms and eukaryotic cells grown in tissue culture.

Recombinant genes may also be introduced into viruses, such as vaccinia virus. Recombinant viruses can be generated by transfection of plasmids into cells infected with virus.

Suitable vectors include, but are not limited to, the following viral vectors such as lambda vector system gt11, gt WES.tB, Charon 4, and plasmid vectors such as pBR322, pBR325, pACYC177, pACYC184, pUC8, pUC9, pUC18, pUC19, pLG339, pR290, pKC37, pKC101, SV 40, pBluescript II SK ± or KS ± (see “Stratagene Cloning Systems” Catalog (1993) from Stratagene, La Jolla, Calif., which is hereby incorporated by reference in its entirety), pQE, pIH821, pGEX, pET series (see F. W. Studier et. al., “Use of T7 RNA Polymerase to Direct Expression of Cloned Genes,” Gene Expression Technology Vol. 185 (1990), which is hereby incorporated by reference in its entirety), and any derivatives thereof. Recombinant molecules can be introduced into cells via transformation, particularly transduction, conjugation, mobilization, or electroporation. The DNA sequences are cloned into the vector using standard cloning procedures in the art, as described by Sambrook et al., Molecular Cloning: A Laboratory Manual, Cold Springs Laboratory, Cold Springs Harbor, N.Y. (1989), which is hereby incorporated by reference in its entirety.

A variety of host-vector systems may be utilized to express the protein-encoding sequence of the present invention. Primarily, the vector system must be compatible with the host cell used. Host-vector systems include but are not limited to the following: bacteria transformed with bacteriophage DNA, plasmid DNA, or cosmid DNA; microorganisms such as yeast containing yeast vectors; mammalian cell systems infected with virus (e.g., vaccinia virus, adenovirus, etc.); insect cell systems infected with virus (e.g., baculovirus); and plant cells infected by bacteria. The expression elements of these vectors vary in their strength and specificities. Depending upon the host-vector system utilized, any one of a number of suitable transcription and translation elements can be used.

Different genetic signals and processing events control many levels of gene expression (e.g., DNA transcription and messenger RNA (“mRNA”) translation).

Transcription of DNA is dependent upon the presence of a promoter which is a DNA sequence that directs the binding of RNA polymerase and thereby promotes mRNA synthesis. The DNA sequences of eukaryotic promoters differ from those of prokaryotic promoters. Furthermore, eukaryotic promoters and accompanying genetic signals may not be recognized in or may not function in a prokaryotic system, and, further, prokaryotic promoters are not recognized and do not function in eukaryotic cells.

Similarly, translation of mRNA in prokaryotes depends upon the presence of the proper prokaryotic signals which differ from those of eukaryotes. Efficient translation of mRNA in prokaryotes requires a ribosome binding site called the Shine-Dalgarno (“SD”) sequence on the mRNA. This sequence is a short nucleotide sequence of mRNA that is located before the start codon, usually AUG, which encodes the amino-terminal methionine of the protein. The SD sequences are complementary to the 3′-end of the 16S rRNA (ribosomal RNA) and probably promote binding of mRNA to ribosomes by duplexing with the rRNA to allow correct positioning of the ribosome. For a review on maximizing gene expression see Roberts and Lauer, Methods in Enzymology, 68:473 (1979), which is hereby incorporated by reference in its entirety.

Promoters vary in their “strength” (i.e., their ability to promote transcription). For the purposes of expressing a cloned gene, it is desirable to use strong promoters in order to obtain a high level of transcription and, hence, expression of the gene. Depending upon the host cell system utilized, any one of a number of suitable promoters may be used. For instance, when cloning in E. coli, its bacteriophages, or plasmids, promoters such as the T7 phage promoter, lac promoter, trp promoter, recA promoter, ribosomal RNA promoter, the P_{R} and P_{L} promoters of coliphage lambda and others, including but not limited, to lacUV5, ompF, bla, lpp, and the like, may be used to direct high levels of transcription of adjacent DNA segments. Additionally, a hybrid trp-lacUV5 (tac) promoter or other E. coli promoters produced by recombinant DNA or other synthetic DNA techniques may be used to provide for transcription of the inserted gene.

Bacterial host cell strains and expression vectors may be chosen which inhibit the action of the promoter unless specifically induced. In certain operons, the addition of specific inducers is necessary for efficient transcription of the inserted DNA. For example, the lac operon is induced by the addition of lactose or IPTG (isopropylthio-beta-D-galactoside). A variety of other operons, such as trp, pro, etc., are under different controls.

Specific initiation signals are also required for efficient gene transcription and translation in prokaryotic cells. These transcription and translation initiation signals may vary in “strength” as measured by the quantity of gene specific messenger RNA and protein synthesized, respectively. The DNA expression vector, which contains a promoter, may also contain any combination of various “strong” transcription and/or translation initiation signals. For instance, efficient translation in E. coli requires a Shine-Dalgarno (“SD”) sequence about 7-9 bases 5′ to the initiation codon (ATG) to provide a ribosome binding site. Thus, any SD-ATG combination that can be utilized by host cell ribosomes may be employed. Such combinations include but are not limited to the SD-ATG combination from the cro gene or the N gene of coliphage lambda, or from the E. coli tryptophan E, D, C, B or A genes. Additionally, any SD-ATG combination produced by recombinant DNA or other techniques involving incorporation of synthetic nucleotides may be used.

Depending on the vector system and host utilized, any number of suitable transcription and/or translation elements, including constitutive, inducible, and repressible promoters, as well as minimal 5′ promoter elements may be used.

The nucleic acid molecule(s) of the present invention, a promoter molecule of choice, a suitable 3′ regulatory region, and if desired, a reporter gene, are incorporated into a vector-expression system of choice to prepare the nucleic acid construct of present invention using standard cloning procedures known in the art, such as described by Sambrook et al., Molecular Cloning: A Laboratory Manual, Third Edition, Cold Spring Harbor: Cold Spring Harbor Laboratory Press, New York (2001), which is hereby incorporated by reference in its entirety.

In one aspect of the present invention, a nucleic acid molecule encoding a protein of choice is inserted into a vector in the sense (i.e., 5′→3′) direction, such that the open reading frame is properly oriented for the expression of the encoded protein under the control of a promoter of choice. Single or multiple nucleic acids may be ligated into an appropriate vector in this way, under the control of a suitable promoters, to prepare a nucleic acid construct of the present invention. In another aspect, the nucleic acid molecule is inserted into the expression system or vector in the antisense (i.e., 3′→5′) orientation.

Once the isolated nucleic acid molecule encoding the LATS2b or LATS2c protein or polypeptide has been cloned into an expression system, it is ready to be incorporated into a host cell. Recombinant molecules can be introduced into cells via transformation, particularly transduction, conjugation, lipofection, protoplast fusion, mobilization, particle bombardment, or electroporation. The DNA sequences are cloned into the host cell using standard cloning procedures known in the art, as described by Sambrook et al., Molecular Cloning: A Laboratory Manual, Second Edition, Cold Springs Laboratory, Cold Springs Harbor, N.Y. (1989), which is hereby incorporated by reference in its entirety. Suitable hosts include, but are not limited to, bacteria, virus, yeast, fungi, mammalian cells, insect cells, plant cells, and the like.

Accordingly, another aspect of the present invention relates to a method of making a recombinant cell. Basically, this method is carried out by transforming a host cell with a nucleic acid construct of the present invention under conditions effective to yield transcription of the DNA molecule in the host cell. Preferably, a nucleic acid construct containing the nucleic acid molecule(s) of the present invention is stably inserted into the genome of the recombinant host cell as a result of the transformation.

Transient expression in protoplasts allows quantitative studies of gene expression since the population of cells is very high (on the order of 10⁶). To deliver DNA inside protoplasts, several methodologies have been proposed, but the most common are electroporation (Neumann et al., “Gene Transfer into Mouse Lyoma Cells by Electroporation in High Electric Fields,” EMBO J. 1: 841-45 (1982); Wong et al., “Electric Field Mediated Gene Transfer,” Biochem Biophys Res Commun 30;107(2):584-7 (1982); Potter et al., “Enhancer-Dependent Expression of Human Kappa Immunoglobulin Genes Introduced into Mouse pre-B Lymphocytes by Electroporation,” Proc. Natl. Acad. Sci. USA 81: 7161-65 (1984, which are hereby incorporated by reference in their entirety) and polyethylene glycol (PEG) mediated DNA uptake, Sambrook et al., Molecular Cloning: A Laboratory Manual, Chap. 16, Second Edition, Cold Springs Laboratory, Cold Springs Harbor, N.Y. (1989), which is hereby incorporated by reference in its entirety). During electroporation, the DNA is introduced into the cell by means of a reversible change in the permeability of the cell membrane due to exposure to an electric field. PEG transformation introduces the DNA by changing the elasticity of the membranes. Unlike electroporation, PEG transformation does not require any special equipment and transformation efficiencies can be equally high. Another appropriate method of introducing the gene construct of the present invention into a host cell is fusion of protoplasts with other entities, either minicells, cells, lysosomes, or other fusible lipid-surfaced bodies that contain the chimeric gene. Fraley, et al., Proc. Natl. Acad. Sci. USA, 79:1859-63 (1982), which is hereby incorporated by reference in its entirety.

Stable transformants are preferable for the methods of the present invention, which can be achieved by using variations of the methods above as describe in Sambrook et al., Molecular Cloning: A Laboratory Manual, Chap. 16, Second Edition, Cold Springs Laboratory, Cold Springs Harbor, N.Y. (1989), which is hereby incorporated by reference in its entirety.

The present invention also relates to an antibody which recognizes the isolated LATS2b protein or polypeptide of the present invention.

Another aspect of the present invention is an antibody which recognizes the isolated LATS2c protein or polypeptide of the present invention.

Antibodies of the present invention include those which are capable of binding to a protein or polypeptide of the present invention and inhibiting the activity of such a polypeptide or protein. The disclosed antibodies may be monoclonal or polyclonal. Monoclonal antibody production may be effected by techniques which are well-known in the art. Monoclonal Antibodies—Production, Engineering and Clinical Applications, Ritter et al., Eds. Cambridge University Press, Cambridge, UK (1995), which is hereby incorporated by reference in its entirety. Basically, the process involves first obtaining immune cells (lymphocytes) from the spleen of a mammal (e.g., mouse) which has been previously immunized with the antigen of interest either in vivo or in vitro. The antibody-secreting lymphocytes are then fused with (mouse) myeloma cells or transformed cells, which are capable of replicating indefinitely in cell culture, thereby producing an immortal, immunoglobulin-secreting cell line. The resulting fused cells, or hybridomas, are cultured, and the resulting colonies screened for the production of the desired monoclonal antibodies. Colonies producing such antibodies are cloned, and grown either in vivo or in vitro to produce large quantities of antibody. A description of the theoretical basis and practical methodology of fusing such cells is set forth in Kohler and Milstein, Nature, 256:495 (1975), which is hereby incorporated by reference in its entirety.

Mammalian lymphocytes are immunized by in vivo immunization of the animal (e.g., a mouse) with the protein or polypeptide of the present invention. Such immunizations are repeated as necessary at intervals of up to several weeks to obtain a sufficient titer of antibodies. Following the last antigen boost, the animals are sacrificed and spleen cells removed.

Fusion with mammalian myeloma cells or other fusion partners capable of replicating indefinitely in cell culture is effected by standard and well-known techniques, for example, by using polyethylene glycol (“PEG”) or other fusing agents. Milstein and Kohler, Eur. J. Immunol., 6:511 (1976), which is hereby incorporated by reference in its entirety. This immortal cell line, which is preferably murine, but may also be derived from cells of other mammalian species, including, but not limited to, rats and humans, is selected to be deficient in enzymes necessary for the utilization of certain nutrients, to be capable of rapid growth, and to have good fusion capability. Many such cell lines are known to those skilled in the art, and others are regularly described.

Procedures for raising polyclonal antibodies are also well known. Typically, such antibodies can be raised by administering the protein or polypeptide of the present invention subcutaneously to New Zealand white rabbits which have first been bled to obtain pre-immune serum. The antigens can be injected at a total volume of 100 μl per site at six different sites. Each injected material will contain synthetic surfactant adjuvant pluronic polyols, or pulverized acrylamide gel containing the protein or polypeptide after SDS-polyacrylamide gel electrophoresis. The rabbits are then bled approximately every two weeks after the first injection and periodically boosted with the same antigen three times every six weeks. A sample of serum is then collected 10 days after each boost. Polyclonal antibodies are then recovered from the serum by affinity chromatography using the corresponding antigen to capture the antibody. Ultimately, the rabbits are euthenized with pentobarbital 150 mg/Kg IV. This and other procedures for raising polyclonal antibodies are disclosed in Harlow, et. al., Eds., Antibodies: A Laboratory Manual, Cold Springs Harbor Laboratory, New York (1988), which is hereby incorporated by reference in its entirety.

It is also possible to use the anti-idiotype technology to produce monoclonal antibodies that mimic an epitope. As used in this invention, “epitope” means any antigenic determinant on an antigen to which the paratope of an antibody binds. Epitopic determinants usually consist of chemically active surface groupings of molecules, such as amino acids or sugar side chains and usually have specific three dimensional structural characteristics, as well as specific charge characteristics. For example, an anti-idiotype monoclonal antibody made to a first monoclonal antibody will have a binding domain in the hypervariable region that is the image of the epitope bound by the first monoclonal antibody.

In addition to utilizing whole antibodies, methods of the present invention encompass use of binding portions of such antibodies. Such binding portions include Fab fragments, F(ab′)₂ fragments, and Fv fragments. These antibody fragments can be made by conventional procedures, such as proteolytic fragmentation procedures, as described in J. Goding, Monoclonal Antibodies: Principles and Practice, pp. 98-118 N.Y. Academic Press (1983), and Harlow et al., Antibodies: A Laboratory Manual, Cold Springs Harbor Laboratory, New York (1988), which are hereby incorporated by reference in their entirety, or other methods known in the art.

Another aspect of the present invention relates to a pharmaceutical composition containing an antibody of the present invention, i.e., an antibody to the LATS2b or LATS2c protein or polypeptide, or an fragment thereof, prepared as described above. The pharmaceutical compositions of the present invention may also include additional components, such as pharmaceutically acceptable adjuvants, carriers, excipients or diluents. In one aspect of the present invention, the pharmaceutical conjugate also includes a cytotoxic component. An exemplary cytotoxic component is ricin. Common toxins used in the construction of immunotoxins include: 1) plant toxins, e.g. ricin, saporin, and PAP (Phytolacca americana pokeweed); 2) bacterial toxins, e.g. Pseudomonas exotoxin (PE) and Diphtheria toxin (DT); and 3) their derivatives. In addition, the conjugate can be a radioisotope conjugate. Examples of radioisotopes used include iodine-131, yttrium-90, iodine-124, copper-64, copper-67, gallium-67, iodine-125, rhenium-188, rhenium-186, bismuth-212, bismuth-213, actinium-225, and astatine-211.

Suitable adjuvants may include, but are not limited to, colloidal aluminum salts (i.e., such as hydroxide, phosphate), lipid A and derivatives, muramyl peptides, saponins, NBP, DDA, cytokines (such as interleukins (1, 2, 3, 6, 12), interferon-γ, tumor necrosis factor), and cholera toxin, B subunit.

Suitable carriers for use in the present invention, may include, but are not limited to, delivery systems, such as emulsions, liposomes, ISCOMS, and micro spheres.

Suitable methods of “administrating” the pharmaceutical conjugates of the present invention include orally, parenterally, subcutaneously, intravenously, intramuscularly, intraperitoneally, by intravesical instillation, by intracavitary, intravesical instillation, intraocularly, intraarterially, intralesionally, or by application to mucous membrane.

Where a non-parenteral introduction mode is selected for use in the present invention, certain preferred embodiments will comprise oral introduction of the pharmaceutical composition into a subject, such as a mammal. Oral administration of the present invention may be achieved by controlled release preparation(s), and sublingual administration.

The present invention also relates to a first method of detecting the expression of LATS2b or LATS2c in a biological sample. This method involves providing an antibody or binding portion thereof that recognizes the LATS2 polypeptide or protein of the present invention and contacting the antibody or binding portion thereof with a biological sample, and detecting any binding that occurs between the biological sample and the antibody or binding portion thereof, thereby detecting the expression of LATS2b or LATS2c in the biological sample. The antibodies of this aspect are prepared as described above using LATS2b or LATS2c as the antigen. Biological samples suitable for use in this aspect of the present invention include body fluid, including, but not limited to, blood, urine, and sperm; and tissue or cells derived from, without limitation, bone marrow, brain, heart, kidney, spleen, thymus, liver, stomach, small intestine, lung, testis, and skin.

Generally, an antibody, or binding portion thereof, is bound to a label effective to permit detection of the cells or tissues upon binding of the biological agent to the cells or tissues. The biological sample is contacted with the antibody, or binding portion thereof, having a label, under conditions effective to permit binding of the antibody or portion thereof to the LATS2b and/or LATS2c protein or polypeptide present in the biological sample.

Examples of labels useful in accordance with the present invention are radiolabels such as ¹³¹I, ¹¹¹In, ¹²³I, ⁹⁹mTc, ³²P, ¹²⁵I, ³H, ¹⁴C, and ¹⁸⁸Rh, fluorescent labels such as fluorescein and rhodamine, nuclear magnetic resonance active labels, chromophores, chemiluminescers such as luciferin, and biologically active enzyme markers such as peroxidase or phosphatase. The antibody or binding portion thereof or probe can be labeled with such reagents using techniques known in the art. For example, see Wensel and Meares, Radioimmunoimaging and Radioimmunotherapy, Elsevier, New York (1983), which is hereby incorporated by reference in its entirety, for techniques relating to the radiolabeling of antibodies. See also, D. Colcher et al., “Use of Monoclonal Antibodies as Radiopharmaceuticals for the Localization of Human Carcinoma Xenografts in Athymic Mice”, Meth. Enzymol. 121: 802-816 (1986); “Cancer Therapy with Radiolabeled Antibodies,” D. M. Goldenberg, Ed., CRC Press, Boca Raton, Fla. (1995), which are hereby incorporated by reference in their entirety.

As will be appreciated by those in the art, “contacting” conditions will be dictated by choice of source sample, e.g., body fluid, tissue, cells, and the method of detection to be used. Binding of a LATS2b and/or LATS2c antibody or fragment thereof to its respective protein or polypeptide in the biological sample is ascertained by detection of the label, thereby indicating the expression of the LATS2b or LATS2c protein or polypeptide in that biological sample.

Detection of antibody binding may be carried out using any of the several methods commonly used for determination of antibody binding, including, but not limited to, western blot, immunoassays, ELISA assay, flow cytometry, radiography, immunoscintography and other diagnostic imaging methods. As will be understood by those skilled in the art, these methods of detection can be utilized as “plus-minus”, i.e., showing a presence or absence of expression, or they may be used for quantitative analysis of protein expression when appropriate standards and positive controls are included.

In one embodiment of this aspect, the labeled antibody or binding portion thereof is administered to a subject in vivo, and the binding of the LATS2b and/or LATS2c antibody or binding portion thereof is directed to a tissue within the subject as is known in the art, and detection of binding to its respective protein or polypeptide is carried by an appropriate diagnostic imaging technique. Suitable detection methods include, without limitation, ultrasound, computed tomography (CT), magnetic resonance imaging (MRI), functional magnetic resonance imaging (fMRI), computed radiography, fluoroscopic radiography, nuclear medicine imaging, and confocal microscopy. Suitable tissues for targeting detection of LATS2b and/or LATS2c include, without limitation: bone marrow; brain, heart, kidney, spleen, thymus, liver, stomach, small intestine, lung, testis, and skin. Subjects may be any mammal, including humans.

Another aspect of the present invention relates to a second method of detecting the expression of LATS2b and/or LATS2c protein or polypeptide in a biological sample. This method involves providing a nucleic acid molecule that specifically hybridizes to a gene encoding a LATS2b, or LATS2c, polypeptide or protein, a probe thereto or primers derived therefrom, and contacting the nucleic acid molecule encoding a LATS2b or LATS2c polypeptide or protein, a probe thereto or primers derived therefrom with a biological sample, and detecting whether the nucleic acid molecule has undergone any hybridization, thereby detecting LATS2b or LATS2c expression in the biological sample. Nucleic acid molecules suitable for this aspect of the present invention include oligonucleotide sequences derived from the appropriate encoding DNA, DNA and RNA complementary to the encoding sequence, complementary oligoribonucleotides such as primers or probes, or oligodeoxyribonucleotides.

Detection of LATS2b and LATS2c expression using nucleic acids molecules can be carried out by a variety of methods known to those in the art, including, but not limited to: Northern blot, Southern blot, PCR, in situ hybridization, and in situ PCR. These and other detection methods are known to those in the art, for example, as described in Sambrook et al., Molecular Cloning: A Laboratory Manual, Second Edition, Cold Springs Laboratory, Cold Springs Harbor, N.Y. (1989); Haimes and Higgins, Nucleic Acid Hybridization: A Practical Approach, IRL Press Limited, Oxford England (1985); Non Radioactive In Situ Hybridization Application Manual, Boehringer Mannheim GmbHY, Biochemica, Mannheim, Germany (1992), which are hereby incorporated by reference in their entirety. As will be understood by those skilled in the art, these methods of detection can be utilized as “plus-minus”, i.e., showing a presence or absence of expression, or they may be used for quantitative analysis of protein expression when appropriate standards and positive controls are included.

The present invention also relates to a method of treating a disease condition in a subject. This method involves providing a therapeutic amount of a pharmaceutical conjugate having an antibody against a LATS2b or LATS2c protein or polypeptide and a cytotoxic component, and administering the conjugate to a subject under conditions effective to form an immune complex with a LATS2b or LATS2c polypeptide or protein, thereby treating a disease condition.

In this aspect of the present invention the antibody of choice, prepared as described above, is administered under conditions effective to form an immune complex with any LATS2b or LATS2c protein or polypeptide present in the biological sample. The cytotoxic component is active/released at the site of the immune complex, and destroys or disables the cells it is in contact with. In this aspect the subject may be any mammal, including, without limitation, a human. An exemplary disease condition to which this aspect of the present invention relates is any cancer in a mammal, including, but not limited to cancers of the soft tissues, bone cancer and leukemia.

The present invention also relates to a method of regulating LATS2b or LATS2c expression in a subject. This method involves administering an antisense nucleic acid molecule of the present invention that is complementary to, and therefore specifically hybridizes to, a nucleic acid molecule of the present invention that encodes a LATS2b or LATS2c protein or polypeptide. Alternatively, this method can be carried out by administering the expression vector of the present invention that contains a LATS2b or LATS2c antisense nucleic acid, prepared as described above. Administering is carried out as described above.

The present invention also relates to a method of gene therapy. This method involves administering to a subject a nucleic acid molecule of the present invention encoding a LATS2b or LATS2c protein or polypeptide, or a fragment thereof, or a vector expressing a LATS2b or LATS2c protein or polypeptide of the present invention.

Gene therapy is a relatively new approach to treatment of diseases. Currently, gene therapy protocols relate to therapy of certain carefully chosen disorders, including certain inherited disorders, a number of aggressively fatal cancers, and AIDS (U.S. Pat. No. 6,316,416 to Patierno, which is hereby incorporated by reference in its entirety). The restricted application of gene therapy to a few disorders reflects concerns about the efficacy, safety, and ethical implications of the approach in general, and current techniques in particular. Despite the cautious approach mandated by these concerns, and despite the fact that techniques for carrying out gene therapy are still in an early stage of development, results from the first few trials have been very encouraging, some spectacularly so. It seems certain that gene therapy techniques will improve rapidly and that gene therapies soon will develop into an increasingly important and ubiquitous modality for treating disease (reviewed, for example, in Tolstoshev, Ann. Rev. Pharm. Toxicol. 32: 573-596 (1993) and Morgan et al., Ann. Rev. Biochem. 62:191-217 (1993), which are incorporated by reference herein in their entirety).

“Gene therapy,” as used herein, generally means the use of a nucleic acid molecule, in a cell, to achieve the production of an agent and the delivery of the agent to a cell or tissue, in situ, i.e., in a subject, to produce an anti-proliferative effect. Approaches to genetic therapy currently being developed, which can be used in accordance with this aspect of the present invention, often are grouped into two major categories: ex vivo and in vivo techniques.

Ex vivo techniques generally proceed by removing cells from a patient or from a donor, introducing a polynucleotide into the cells, usually selecting and growing out, to the extent possible, cells that have incorporated, and, most often, can express the polynucleotide, and then introducing the selected cells into the patient. Cells that target tumor cells in vivo, including tumor cells that have migrated from primary or secondary tumor sites, generally are preferred in this type of gene therapy.

In addition, as described further below, a nucleic acid molecule of the present invention may be introduced directly into the subject. The nucleic acid in this case may be introduced systemically or by injection into a tumor site. The nucleic acid may be in the form of DNA or RNA, alone or in a complex, or in a vector.

The nucleic acid molecule may be in any of a variety of forms, for example, a DNA (in either a sense or antisense form), a DNA fragment cloned in a DNA vector, a DNA fragment cloned in DNA vector and encapsidated in a viral capsid, RNA, PNA, or other useful forms for introduction into the subject.

When incorporated into a vector, the nucleic acid construct may include a promoter, enhancer, and other cis-acting control regions that provide a desired level and specificity of expression in the cells of a region operably linked thereto that encodes an RNA, such as an anti-sense RNA, or a protein. The nucleic acid construct may contain several such operably linked control and encoding regions for expression of one or more mRNAs or proteins, or a mixture of the two.

The nucleic acid molecule of the present invention encoding a nucleic acid encoding a LATS2b or LATS2c protein or polypeptide may be introduced into cells either ex vivo or in vivo, including into a tumor in situ. A variety of techniques have been designed to deliver polynucleotides into cells for constitutive or inducible expression, and these routine techniques can be used in gene therapy of the present invention as well. Nucleic acid molecules will be delivered into cells ex vivo using cationic lipids, liposomes or viral vectors. Introduction into cells in vivo, including into cells of tumors in situ, will be using direct or systemic injection. Methods for introducing nucleic acid molecules in this manner can involve direct injection of a nucleotide, which then generally will be in a composition with a cationic lipid or other compound or compounds that facilitate direct uptake of DNA by cells in vivo. Such compositions may also comprise ingredients that modulate physiological persistence. In addition, the nucleic acid molecule can be introduced into cells in vivo in viral vectors.

Genetic therapies in accordance with the present invention may involve a transient (temporary) presence of the gene therapy polynucleotide in the patient or the permanent introduction of a polynucleotide into the patient. In the latter regard, gene therapy may be used to repair a dysfunctional gene to prevent or inhibit metastasis or cellular proliferation. Genetic therapies, like the direct administration of agents discussed above, in accordance with the present invention may be used alone or in conjunction with other therapeutic modalities.

In one aspect of the present invention, the subject of the methods of gene therapy according to the present invention is a mammal, including human and non-human subjects.

The present invention also relates to transgenic animals with altered expressions of LATS2b or LATS2c. In this context, “altered” refers to either up-regulation or down-regulation of the expression of LATS2b and/or LATS2c protein or polypeptide in a subject.

Methods of altering the expression of endogenous proteins by transfer of recombinant genes into cell in culture and into live animals have been developed. For example, DNA molecules have been introduced into cultured cells by calcium phosphate precipitation and electroporation (Graham et al., Virology, 52:456-467 (1973); Perucho et al., Cell 22:9-17 (1980); Chu et al., Nucleic Acids Research 15:1311-1326 (1987); and Bishop and Smith, Molecular Biology Medicine 6:283-298 (1989), which are hereby incorporated by reference in their entirety). DNA molecules have also been introduced into the nucleus of cells in culture by direct microinjection (Gordon et al., Proc. Natl. Acad. Sci. USA 77:7380-7384 (1980); Gordon et al, Methods in Enzymology 101:411-433 (1983); and U.S. Pat. No. 4,873,191, to Wagner et al., which are hereby incorporated by reference in their entirety).

Retroviral vectors have also been used to introduce DNA molecules into the genome of animals (Jaenisch et al., Cell 24:519 (1981); Soriano et al., Science 234:1409-1413 (1986); and Stewart et al., EMBO J. 6:383-388 (1987), which are hereby incorporated by reference in their entirety). Recombinant genes have been introduced into primary cultures of bone marrow, skin, fibroblasts, or hepatic or pancreatic cells, and then transplanted into live animals. Transgenic animals have also been developed as bioreactors for desired biologically active molecules (U.S. Pat. No. 6,339,183 to Sun; U.S. Pat. No. 6,255,554 to Lubon et al., which are hereby incorporated by reference in their entirety).

The term ‘animal’ as used herein denotes all mammalian animals except humans. Farm animals (pigs, goats, sheep, cows, horses, rabbits and the like), rodents (such as mice), and domestic pets (for example, cats and dogs) are included in the scope of this invention. It also includes an individual animal in all stages of development, including embryonic and fetal stages. A “transgenic” animal is any animal containing cells that bear genetic information received, directly or indirectly, by deliberate genetic manipulation at the subcellular level, such as by microinjection or infection with recombinant virus.

“Transgenic” in the present context does not encompass classical crossbreeding or in vitro fertilization, rather, herein denotes animals in which one or more cells receive a recombinant nucleic acid molecule. Although it is highly preferred that this molecule be integrated within the animal's chromosomes, the invention also encompasses the use of extrachromosomally replicating nucleic acid molecule sequences, such as might be engineered into yeast artificial chromosomes.

The term “germ cell line transgenic animal” refers to a transgenic animal in which the genetic information has been taken up and incorporated into a germ line cell, therefore conferring the ability to transfer the information to offspring. If such offspring, in fact, possess some or all of that information, then they, too, are transgenic animals.

The information to be introduced into the animal is preferably foreign to the species of animal to which the recipient belongs (i.e., “heterologous”), but the information may also be foreign only to the particular individual recipient, or genetic information already possessed by the recipient. In the last case, the introduced gene may be expressed differently than is the native gene.

In the context of the present invention, the “up-regulation” of expression of a protein or polypeptide in a transgenic animal would involve the introduction into the animal a nucleic acid construct, in a suitable vector, that includes one or more DNA molecules that encode the desired protein or polypeptide of the present invention, (i.e, either LATS2b or LATS2c). In one aspect, the nucleic acid molecule of the encoded protein is inserted in the vector of choice in a proper sense (5′→3′) orientation and correct reading frame. The vector contains the necessary elements for the transcription and translation of the inserted protein-coding sequences. The nucleic acid molecule of the encoded protein may be under the control of a promoter that provides for the constitutive overexpression of the encoded protein. Alternatively, the construct is under the control of an inducible promoter that can be manipulated externally for expression of the protein or polypeptide when most desirable.

In the context of the present invention, the “down-regulation” of expression of a protein or polypeptide in a transgenic animal would involve the introduction into the animal of a nucleic acid construct, in a suitable vector, that is complementary to the nucleic acid molecule that encodes the desired protein or polypeptide of the present invention. In one aspect, this nucleic acid molecule is an antisense molecule of the encoded protein, as described earlier herein, and under the control of a either a constitutive or inducible promoter. As described above, the antisense nucleic acid will interfere with the normal transcription and/or translation mechanism of the cell, and “block” expression. Alternatively, the nucleic acid may be a “sense” DNA molecule that encodes a LATS2b or LATS2c protein or polypeptide, modified such that the open reading frame is shifted, or otherwise modified, such proper transcription is not possible. In either embodiment, the result may be that protein expression is completely eliminated, or it may be only decreased, as compared with the production of a LATS2b or LATS2c from a non-manipulated LATS2b or LATS2c-producing cell. In this embodiment, overproliferation or hyperproliferation is desirable, for example, to produce larger farm animals.

The present invention also relates to a method of regulating cell growth or differentiation. This method involves introducing to cells a vector expressing a LATS2b or LATS2c nucleic acid molecule, thereby regulating the growth or differentiation of the cells. In this aspect of the present invention, cell growth and differentiation is either up-regulated or down-regulated. Up-regulation is carried out by inhibiting or decreasing the expression of LATS2b or 2c in a cell. Therefore, in one aspect, the vector includes either an antisense LATS2b or 2c nucleic acid molecule, or a LATS2b or 2c nucleic acid molecule that results in the expression of an interfering RNA. The use of antisense and RNA interference technologies to decrease or silence gene expression, including the preparation of antisense and iRNA-expressing vectors is described above. Down-regulation of cell growth and differentiation in this aspect of the present invention is carried out by increasing the expression of LATS2b or 2c in a cell over that expressed in the cell without manipulation. In this aspect, the vector includes a LATS2b or 2c nucleic acid molecule capable of being highly expressed in the cell. Generally, this will involve introducing a vector having into the cell having one or more LATS2b or 2c nucleic acid molecules capable of expression in the cell of choice. The preparation of the vector of this aspect of the present invention is as described detail above. Suitable cells for use in this aspect include, without limitation, hematopoietic cells and stems cells. Introduction of a suitable vector into a cell of choice may be carried out either in vivo or in vitro, using methods described above.

Another aspect of the present invention is a method of altering the expression of LATS2, LATS2b or LATS2c in a cell or subject. This method involves treating a cell with a chemical or molecule capable of interfering with circadian control of the cell, thereby altering the expression of LATS2, 2b or 2c in the cell or subject. As described in greater detail in the Background, supra, and Examples, infra, LATS2 (GenBank accession number BAA92380, which is hereby incorporated by reference in its entirety) is a clock-controlled gene. Therefore, by disrupting, or, resetting the circadian clock of a cell or subject, the expression of clock-controlled genes, including LATS2, 2b, and 2c, can be altered.

Another aspect of the present invention is a method of screening for drugs that regulate LATS2b and/or LATS2c activity. This method involves providing the LATS2b or LATS2c protein or polypeptide of the present invention, a reagent upon which LATS2c or LATS2b is known to exert a biological activity, and a test compound. The LATS2 protein or polypeptide of choice, the reagent, and the test compound are blended to form a mixture under conditions appropriate for the protein or polypeptide to exert its activity upon the reagent. The activity of the LATS2b or LATS2c protein or polypeptide being tested is then determined, and the difference in activity between the activity of the LATS2 protein upon the reagent with and without the test compound is measured. This difference may be measured quantitatively by the use of appropriate LATS2b or LATS2c standards in the test situation.

The present invention also relates to a second method of screening for drugs that regulate the expression of LATS2b and/or LATS2c. This method involves transforming a host cell with a nucleic acid construct having a nucleic acid molecule encoding a LATS2b or LATS2c protein or polypeptide operably linked to transcriptional and translational regulatory elements, culturing the transformed cells, adding a test compound to the culture containing the transformed cells, and determining whether the test compound regulates the expression of LATS2b or LATS2c in the transformed cells.

The transformed cells are cultured in a medium suitable for allowing LATS2 expression, and the drug or test compound is added to the cell culture system. The expression of LATS2b or LATS2c is determined by any of the methods described herein for the detection of protein or polypeptide expression, or by any suitable method known to those in the art.

Any mammalian cell is suitable for this aspect of the present invention, including human cells. Human cells suitable for this aspect of the present invention include, but are not limited to, those derived from bone marrow, brain, heart, kidney, spleen, thymus, liver, stomach, small intestine, lung, testis, and skin.

The present invention also relates to a method of screening for drugs that regulate LATS protein expression which involves isolating cells from a transgenic animal having altered expression of LATS2b or LATS2c, as described above, adding a test compound to the isolated cells under appropriate conditions, and determining whether the test compound regulates the expression of LATS2b or LATS2c in the isolated cells.

### EXAMPLES

### Example 1

Housing of Animals

Male mice (Balb/c, 3-4 weeks old; Jackson Laboratory) were used to avoid interference by the female estral rhythm. Upon arrival, the mice were acclimated in the same room with a 12:12 light-dark cycle for at least two weeks prior to the initiation of the experiments. To diminish the disturbance of the sleep phase, the mice were housed 2 to 3 per cage. At each time point, bone marrow cells were harvested from the mice from one cage. The procedures were performed under a dim light during the dark phase of the light-dark cycle.

### Example 2

Bone Marrow Collection

Mice were sacrificed by cervical dislocation at 0, 4, 8, 12, 16, and 20 hours after light onset. (The light was turned off at 12 hours after light onset.) The femurs of individual mice were removed and the bone marrow cells were flushed with McCoy's 5A medium (Gibco, Grand Island, N.Y.) supplemented with 1% fetal bovine serum (FBS) (Hyclone, Logan, Utah). The bone marrow cells collected at each time point were lysed with the lysis buffer RLT (Qiagen, Valencia, Calif.) and stored at −70° C.

### Example 3

RNA Arbitrarily Primed PCR (RAP-PCR)

Total RNA was purified from the bone marrow cells using the RNeasy Mini Kit (Qiagen, Valencia, Calif.) according to the manufacturer's instructions. RAP-PCR was performed using RAP-PCR kit (Stratagene, La Jolla, Calif.) following the manufacturer's protocol. Following DNase (Promega, Madison, Wis.) treatment, 1 μg total RNA was used to synthesize first-strand cDNA with the random primer A2 (Stratagene, La Jolla, Calif.) at 37° C. for 60 minutes. A quarter of the cDNA was used for PCR. The same random primer was used for PCR at the following conditions. The first cycle at 94° C. for 1 minute, 36° C. for 5 minutes, and 72° C. for 5 minutes, followed by 40 cycles at 94° C. for 1 minute, 52° C. for 2 minutes, and 72° C. for 2 minutes. PCR products were resolved on 7M urea, 6% acrylamide gels and visualized by silver stain solution (Pharmacia, Piscataway, N.J.). Differentially displayed bands were excised, extracted from the gel, amplified, cloned, and sequenced. The DNA sequences were then compared to the various databases at GenBank.

### Example 4

Relative Quantitative Reverse Transcriptase-Polymerase Chain Reaction (RT-PCR)

For each RT-PCR experiment, samples from six time points were analyzed at the same time. Total RNA was purified from 2×10⁶ bone marrow cells using the RNeasy Mini Kit (Qiagen) according to the manufacturer's protocol. One sixth of the total RNA was reverse transcribed using Moloney murine leukemia virus reverse transcriptase (MMLV-RT; Stratagene, La Jolla, Calif.) with random primers (Stratagene, La Jolla, Calif.) at 37° C. for 60 minutes in a 20-μl reaction. The internal control (Quantum RNA 18S Internal Standards; Ambion) was used according to the manufacturer's protocol to analyze the relative amounts of the indicated mRNA at different time points. The 18S non-productive competing primers (Competimer; Ambion, Austin, Tex.) are designed to carry modified 3′ ends for blocking the extension by DNA polymerase. A 9:1 ratio of the 18S non-productive competing primers to the 18S primer mix was used to reduce the 18S cDNA signal to a level comparable to that of the target gene. The 18S cDNA and target cDNA (6A-2-9, mlats2, or mlats2b) were coamplified in a PCR-tube. Primers (see Table 1, below) used were Forward Primer 1 and Reverse Primer 4 for clone 6A-2-9; Forward Primer 1 and Reverse Primer 1 for mlats2; and Forward Primer 1 and Reverse Primer 2 for mlats2b. Within each PCR experiment, the linear range of amplification was first determined using cDNA pooled from 6 time points. PCR was performed with Taq DNA polymerase (Advantage cDNA Polymerase Mix; CLONTECH, Palo Alto, Calif.) in 1× PCR reaction buffer (CLONTECH, Palo Alto, Calif.) containing 0.8 mM dNTPs under the following conditions: initial incubation at 94° C. for 3 minutes, 25-30 cycles (depending on the linear range) at 94° C. for 30 seconds, 58° C. (for 6A-2-9 and mlats2) or 62° C. (for mlats2b) for 30 seconds and 72° C. for 30 seconds, followed by a 7-minute extension at 72° C. As a negative control, the products of the RT reactions, without reverse transcriptase, were subjected to the same PCR amplification. The PCR products were resolved by electrophoresis on a 1.5% agarose gel (Gibco) and stained with the fluorescent stain (GelStar; FMC, Rockland, Me.). Their relative quantities were then determined by using the Image-Pro Plus software (Media Cybernetics).

TABLE 1

| | | | | |
|---|---|---|---|---|
| Primer | Sequence | SEQ ID NO: | Nucleotide Positions | Reference |

| | | | | |
|---|---|---|---|---|
| Forward Primer 1 | 5′ AAGGAAACTGGACTAACAATGAGGC 3′ | 14 | 116 to 140 in mlats2 | GenBank AB023958 |
| Forward Primer 2 | 5′ CACTGACACTGTTGACTGTTCTCT 3′ | 15 | 50 to 63 in mlats2 | GenBank AB023958 |
| Reverse Primer 1 | 5′ GGTCTGCTTGATGACTCGCACAATC 3′ | 16 | 574 to 598 in mlats2 | GenBank AB023958 |
| Reverse Primer 2 | 5′ GACACGCACCAGGAATATGCATCTG 3′ | 17 | 421 to 445 in mlats2b | |
| Reverse Primer 3 | 5′ ACACGCACCAGGAATATGCATTGT 3′ | 18 | 424 to 444 in mlats2b and | |
| | | | 145 to 147 in the insertion | |
| | | | of mlats2c | |

### Example 5

3′-Rapid Amplification of the cDNA End (RACE)

Total RNA was purified from the bone marrow cells using the RNeasy Mini Kit (Qiagen, Valencia, Calif.) according to the manufacturer's instructions. 3′-rapid amplification of the cDNA end (RACE) was carried out using the SMART RACE cDNA Amplification Kit (CLONTECH, Palo Alto, Calif.) as suggested by the manufacturer. Briefly, the first-strand cDNA was synthesized using a primer containing a stretch of oligo (dT) and a universal primer binding sequence (CLONTECH, Palo Alto, Calif.). PCR was carried out using the Forward Primer 1 (Table 1) and the universal primer (CLONTECH, Palo Alto, Calif.) as follows: 5 cycles each at 94° C. for 5 seconds and 72° C. for 3 minutes; followed by 5 cycles each at 94° C. for 5 seconds, 70° C. for 10 seconds, and 72° C. for 3 minutes and 30 cycles each at 94° C. for 5 seconds, 68° C. for 10 seconds, and 72° C. for 3 minutes. The PCR product was cloned into the pCRII-TOPO TA cloning vector (Invitrogen, Carlsbad, Calif.) and its sequence determined by the dye terminator cycle sequencing method using a model 373 AD DNA sequencer (Applied Biosystems).

### Example 6

Reverse Transcriptase-Polymerase Chain Reaction (RT-PCR)

Following DNase (Promega, Madison, Wis.) treatment, approximately 2 μg of total RNA from mouse bone marrow cells was reverse transcribed using Moloney murine leukemia virus reverse transcriptase (MMLV-RT; Stratagene, La Jolla, Calif.) with random primers (Stratagene, La Jolla, Calif.) in a 20-μl reaction. The resulting reaction mixture (2.5 μl) was used as a PCR template in a 25-μl reaction using Taq DNA polymerase (AdvanTaq Plus DNA Polymerase; Clontech, Palo Alto, Calif.) under the following conditions: initial incubation at 94° C. for 3 minutes, 35 cycles each at 94° C. for 10 seconds, 58° C. for 30 seconds and 72° C. for 30 seconds, and the final incubation at 72° C. for 7 minutes. Primers (Table 1) used were Forward Primer 1 and Reverse Primer 1 for mlats2, Forward Primer 1 and Reverse Primer 2 for mlats2b and Forward Primer 2 and Reverse Primer 3 for mlats2c.

### Example 7

PCR Analysis of Gene Expression in Different Mouse Tissues

A PCR-based method was used to analyze the expression profiles of mlats2, mlats2b, and mlats2c in different mouse tissues using the RAPID-SCAN Gene Expression Panel (OriGene). According to the manufacturer, the expression panel was prepared by isolating total RNA from different tissues of adult Swiss Webster mice. Poly-A⁺ RNA was then isolated and subjected to the first-strand cDNA synthesis using an oligo(dT) primer. Individual cDNA pools were confirmed to be free of genomic DNA contamination. For analysis of mlats2, mlats2b, and mlats2c expression, 1 ng of cDNA was used as the template for each tissue. The primer sets specific for individual splice variants are the same as described above. mlats2 and mlats2b were coamplified in the same PCR tubes. The PCR conditions were the same as described above for RT-PCR. For β-actin, 1 pg of cDNA from each tissue and the β-actin primer set (OriGene) were used as suggested by the manufacturer.

### Example 8

Plasmid Construction

pcDNA3-mLATS2 and pcDNA3-mLATS2N373 were generated by inserting the entire mLATS2 open reading frame (kindly provided by Dr. Hiroshi Nojima at Osaka University, Japan) or the BamH I-Not I fragment into the BamH I and Xho I sites or BamH I and Not I sites of pcDNA3 (Invitrogen, Carlsbad, Calif.), respectively. pGBKT7-mLATS2b was constructed by inserting the PCR-generated entire coding region of mlats2b into the Nde I and Sma I sites of pGBKT7 (CLONTECH, Palo Alto, Calif.) in frame to the GAL4 DNA binding domain. The same PCR product was cloned into pcDNA3 to create pcDNA3-mLATS2b. pGBKT7-mLATS2 was generated by inserting the Bsm I-Xho I fragment of pcDNA3-mLATS2 into the Bsm I and Sal I sites of pGBKT7-mLATS2b. pGBKT7-mLATS2N373 was constructed by removing the Not I fragment from pGBKT7-mLATS2. pGBKT7-mLATS2N96 was constructed by removing the Pst I fragment from pGBKT7-mLATS2b. The coding region of mRBT1 was PCR-amplified and cloned into the EcoR I and Pst I sites of pM (CLONTECH, Palo Alto, Calif.) in frame to the GAL4 DNA binding domain to generate pM-mRBT1. The same PCR product was cloned into the EcoR I and Sma I sites of pGADT7 (CLONTECH, Palo Alto, Calif.) in frame to the activation domain to create pGADT7-mRBT1. pGADT7-mRBT1N121 was generated by removing the Xho I fragment from pGADT7-mRBT1. The PCR product encoding the C-terminal 76 amino acids of mRBT1 was cloned into the EcoR I and Sma I sites of pGADT7 to create pGADT7-mRBT1C76. The same PCR product was cloned into the EcoR I and Pst I sites of pM to generate pM-mRBT1C76. pG5-E1b-LUC, in which 5 GAL4-binding sites and the E1b-minimal promoter are located upstream of the luciferase gene, was constructed as previously described (Hsiao et al., “The Linkage of Kennedy's Neuron Disease to ARA24, the First Identified Androgen Receptor Polyglutamine Region-Associated Coactivator,” J Biol Chem 274(29):20229-34 (1999), which is hereby incorporated by reference in its entirety).

### Example 9

Yeast Two-Hybrid Assay

Yeast two-hybrid screening was performed using the MATCHMAKER GAL4 Two-Hybrid System 3 (CLONTECH, Palo Alto, Calif.) and a human bone marrow MATCHMAKER cDNA library purchased from CLONTECH (Palo Alto, Calif.) according to the manufacturer's instructions. Competent cells (AH109) were prepared as follows. 2 ml of the YPD medium was inoculated with a single colony and incubated overnight at 30° C. with shaking. 100 μl of the overnight culture was transferred into 25 ml of the YPDA medium and grown overnight at 30° C. with shaking to the stationary phase. The overnight culture was then transferred into 150 ml of the YPDA medium and grown for additional 2 to 3 hours. Cells were harvested and washed once with 35 ml of sterile water. Finally, cells were resuspended in 0.75 ml 1× TE/LiAc solution. Cells were transformed with the bait and library plasmids as described in the manufacturer's manual. After transformation, cells were plated on quadruple dropout plates (-Ade/-His/-Leu/-Trp) to select for positive protein-protein interactions. Clones grown on the quadruple dropout plates were further confirmed by X-alpha-Gal (CLONTECH, Palo Alto, Calif.) as appearance of blue colonies. The inserts of the positive clones were sequenced using a DNA sequencer (Perkin-Elmer ABI 377).

### Example 10

Mammalian One-Hybrid Assay

NIH 3T3 mouse fibroblast cells were maintained in DMEM supplemented with 10% FBS (Hyclone, Logan, Utah). The daybefore transfection, 3×10⁵ cells/well were plated onto six-well plates. Cells were transfected with indicated amounts of the expression plasmid(s), 100 ng of pG5-E1b-LUC, and 4 ng of the Renilla luciferase control plasmid (pRL-SV40; Promega, Madison, Wis.) using SuperFect transfection reagent (Qiagen, Valencia, Calif.). The Renilla luciferase control plasmid was cotransfected to normalize transfection efficiency. The total amount of the plasmids was adjusted by adding the pcDNA3 plasmid to 1.6 μg/well. Forty hours after transfection, cells were washed once with phosphate-buffered saline (PBS; Gibco, Grand Island, N.Y.) and lysed with 500 μl of passive lysis buffer (Promega, Madison, Wis.). Luciferase activity was assayed with the Dual-Luciferase Reporter Assay System (Promega, Madison, Wis.) using a luminometer (Optocomp1; MGM Instruments) as recommended by the manufacturer.

### Example 11

Southern Blot Analysis

Mouse genomic DNA was purified from the bone marrow cells by the Genomic-tip 500 column (Qiagen, Valencia, Calif.) following the manufacturer's instructions. Genomic DNA (10 μg) was digested with Pst I and separated on a 0.8% agarose gel. DNA was then transferred onto a positive-charged nylon membrane (Boehringer Mannheim) through capillary action. Southern blot analysis was performed using a digoxigenin-labeled probe generated by PCR (PCR DIG Probe Synthesis Kit; Boehringer Mannheim) following manufacturer's protocol. Briefly, the membrane was blocked with blocking solution (Boehringer Mannheim) for 2 hours at 42° C. Hybridization was carried out at 42° C. overnight with DIG Easy Hyb hybridization buffer (Boehringer Mannheim) containing digoxigenin-labeled probes at a final concentration of 25 ng/ml. After hybridization, the membrane was washed twice, 5 minutes each, with 2× wash solution (2× SSC and 0.1% SDS) at room temperature, followed by additional two washes, 5 minutes each, with 0.5× wash solution (0.5× SSC and 0.1% SDS) at 68° C. Detection was performed using alkaline phosphatase-conjugated anti-digoxigenin antibodies and the chemiluminescent substrate CSDP (Boehringer Mannheim).

### Example 12

Identification of mlats2 as a Potential Clock-Controlled Gene and Cloning of its Two Novel Splice Variants

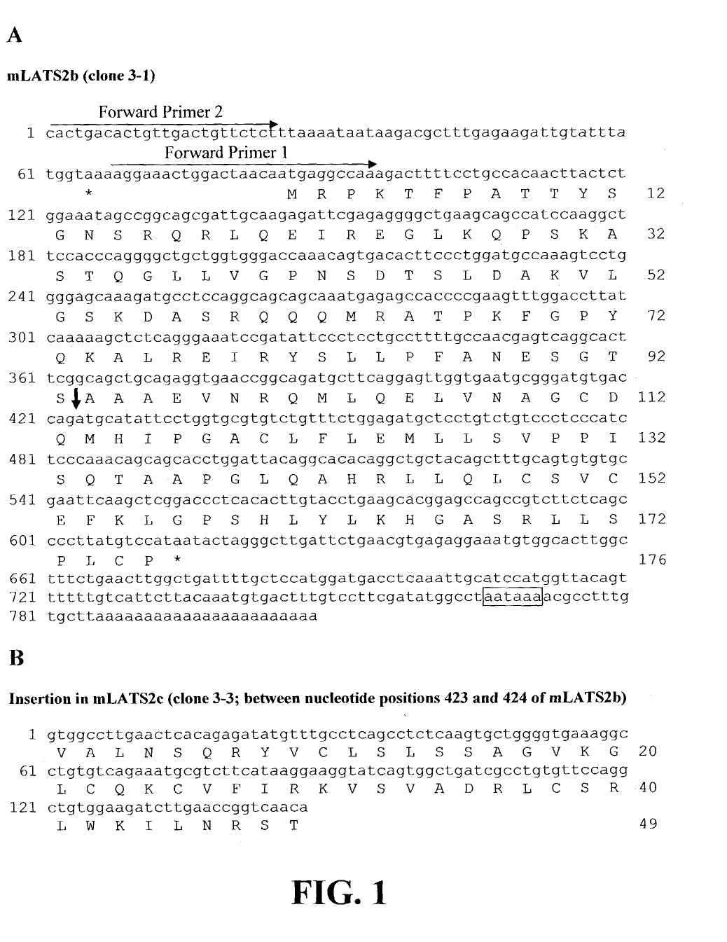

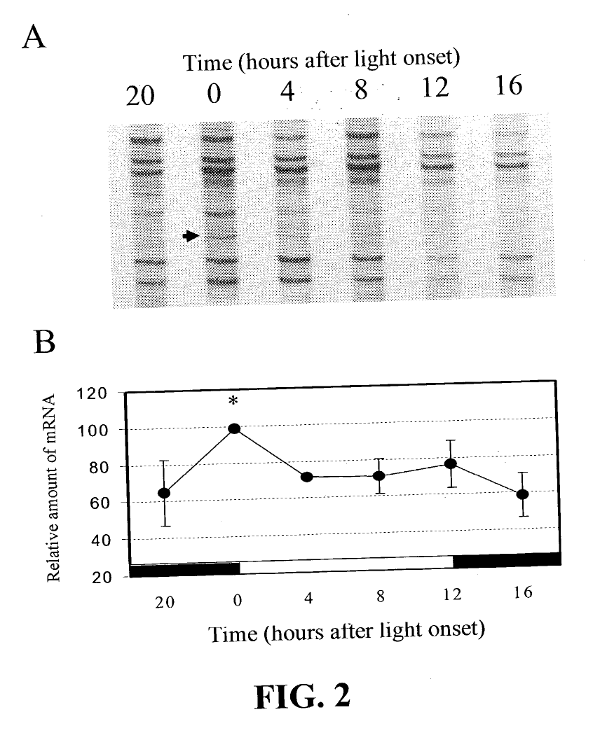

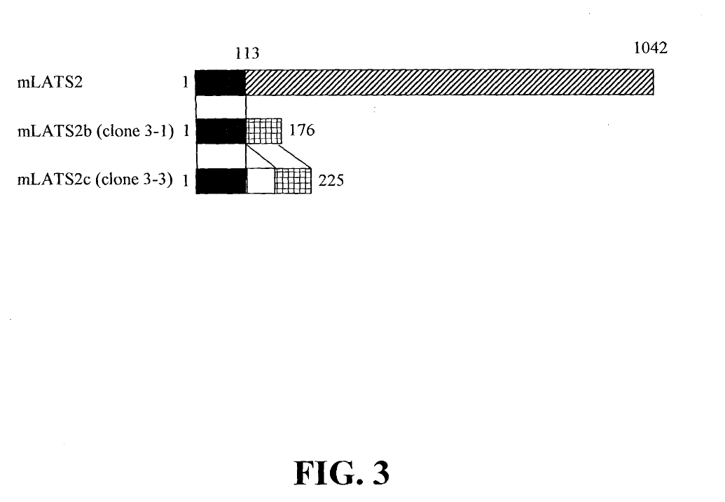

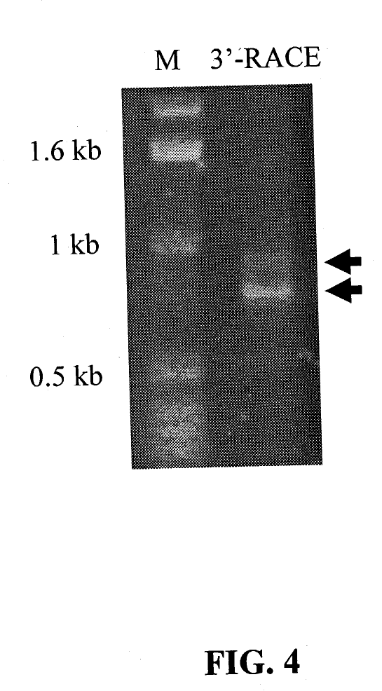

Total murine bone marrow cells were collected at 6 different times of the day for direct comparison of gene expression patterns using the RNA arbitrarily primed PCR (RACE) technique. DNA bands that showed circadian oscillation were excised from the gel for determination of their sequences. A cDNA (6A-2-9) encoding a polypeptide homologous to a cell cycle regulator hLATS1 was cloned, shown in FIG. 2A. The circadian expression pattern of 6A-2-9 was confirmed by relative quantitative RT-PCR, as shown in FIG. 2B. The open reading frame of 6A-2-9 contains a putative start codon but the 3′ end is not complete. In the attempt to clone full-length cDNA of this gene using the 3′-RACE technique employing a primer corresponding to the putative start codon (Forward Primer 1, Table 1) revealed two distinct cDNA fragments. Two PCR products of about 750 and 890 base pairs, respectively, were obtained. Subsequently, it was found that the cDNA clone 6A-2-9 indeed codes for part of mLATS2 (Yabuta et al., “Structure, Expression, and Chromosome Mapping of LATS2, a Mammalian Homologue of the Drosophila Tumor Suppressor Gene Lats/Warts,” Genomics 63(2):263-70 (2000), which is hereby incorporated by reference in its entirety). However, the 3′-RACE products indicated by the arrows in FIG. 4 are much shorter than the reported mlats2 cDNA (>3000 bp). The first 357 base pairs (nucleotides 67-423, FIG. 1A) of the original cloned 3′-RACE products, namely clones 3-1 and 3-3, are identical to the 5′ region of mlats2 (nucleotides 116 to 472, GenBank accession number AB023958, which is hereby incorporated by reference in its entirety). The 5′ identical region between mlats2 and clone 3-1/3-3 was further extended (nucleotides 1-66 in FIG. 1A) by PCR employing Forward Primer 2 paired with Reverse Primer 2 or Reverse Primer 3 (Table 1). The poly-adenylation signal AATAAA is found 14 bp upstream from the poly-A tail, shown in the box in FIG. 1A. When compared to mLATS2 (GenBank accession number BAA92380, which is hereby incorporated by reference in its entirety), the deduced amino acids of clones 3-1 and 3-3 contain the same N-terminal 113 residues as those of mLATS2 but distinct C-termini, shown in FIG. 3. Furthermore, clone 3-3 contains an in-frame insertion of 49 amino acids not found in mLATS2 or clone 3-1.

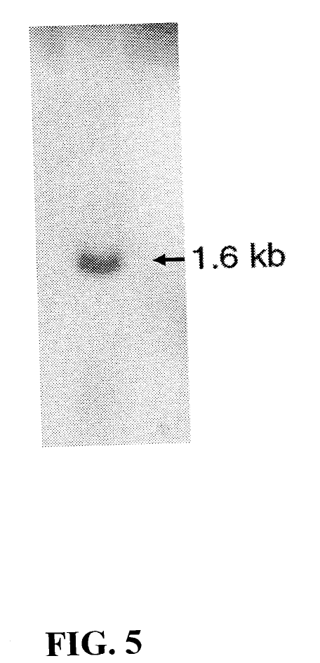

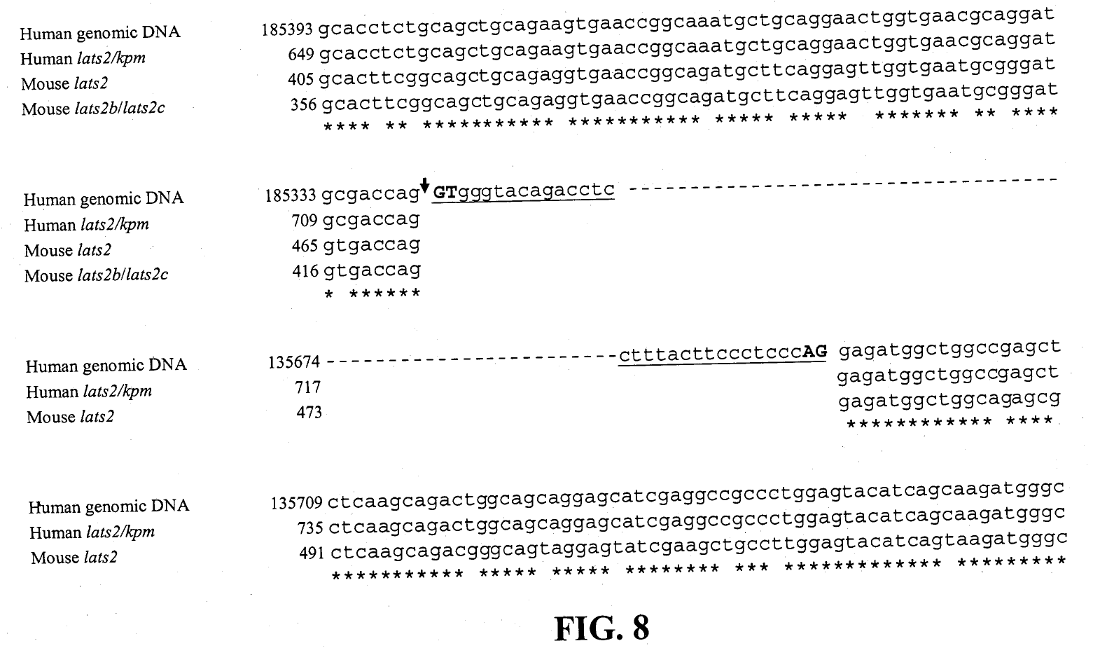

Homology search using the BLAST program revealed that at least clone 3-1 has been identified independently (two EST clones; GenBank accession numbers AA821553 and BF147719, which are hereby incorporated by reference in their entirety). Sequence alignment among mlats2, hlats2/kpm, clones 3-1/3-3, and the corresponding human genomic DNA sequence (GenBank accession number NT_{—}009917, which are hereby incorporated by reference in their entirety), shows a putative intron located at between nucleotides 716 and 717 of hlats2/kpm, shown in FIG. 8. The putative splice site corresponds to nucleotides 423 and 424 of clones 3-1/3-3, respectively, representing the exact location where the identity between mlats2 and clones 3-1/3-3 breaks off (shown as short arrow in FIG. 1A). The putative splice donor and acceptor in the human genomic DNA conform to the GT/AG rule (Stephens et al., “Features of Spliceosome Evolution and Function Inferred From an Analysis of the Information at Human Splice Sites,” J Mol Biol 228(4):1124-36 (1992), which is hereby incorporated by reference in its entirety). Because the nucleotide sequences of mlats2 and hlats2/kpm are well conserved in this region, it is most likely that nucleotides 472 and 473 of mlats2 (GenBank accession number AB023958, which is hereby incorporated by reference in its entirety); corresponding to nucleotides 423 and 424 of clones 3-1/3-3, respectively) are also at the exon-intron boundaries. In addition, the fact that the 5′ regions, including a portion of the 5′ untranslated region (5′ UTR), in all three transcripts are identical further supports that clones 3-1 and 3-3 are derived from alternative splicing of the mlats2 gene. To further ascertain that mlats2 is a single copy gene in the mouse genome, Southern blot analysis was carried out using a probe within the region common to mlats2, clone 3-1 and clone 3-3 (nucleotides 67 to 389 in clone 3-1; FIG. 1A). Based on the comparison between human genomic DNA and the mlats2 cDNA, it appears that the sequence covered by the probe is located in one exon. Therefore, a single band should be obtained on the Southern blot if mlats2, clone 3-1, and clone 3-3 are derived from the same gene. As shown in FIG. 5, a single band of about 1.6 kb was observed. In addition, the mlats2 gene has been located in the central region of mouse chromosome 14 by interspecific mouse backcross mapping (Yabuta et al., “Structure, Expression, and Chromosome Mapping of LATS2, a Mammalian Homologue of the Drosophila Tumor Suppressor Gene Lats/Warts,” Genomics 63(2):263-70 (2000), which is hereby incorporated by reference in its entirety). All taken together, it therefore appears that clones 3-1 and 3-3 are the alternatively spliced forms of mlats2. These two novel splice variants are hereafter named mlats2b (GeneBank Accession No. AY015061) and mlats2c (GeneBank Accession No. AY015062), respectively.

### Example 13

Expression of the lats2 Splice Variants in Different Mouse Tissues

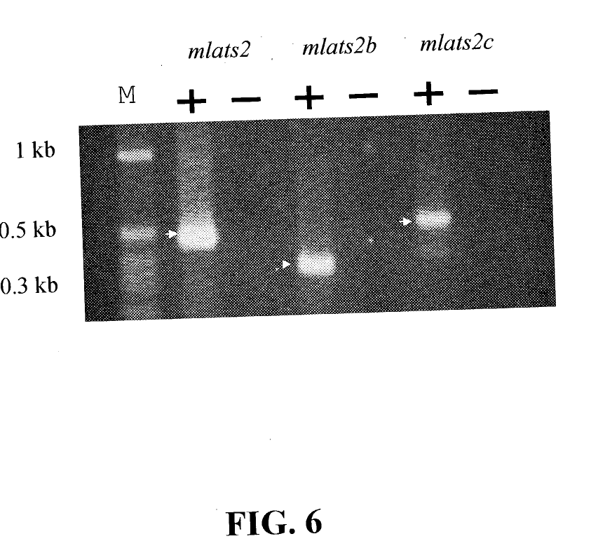

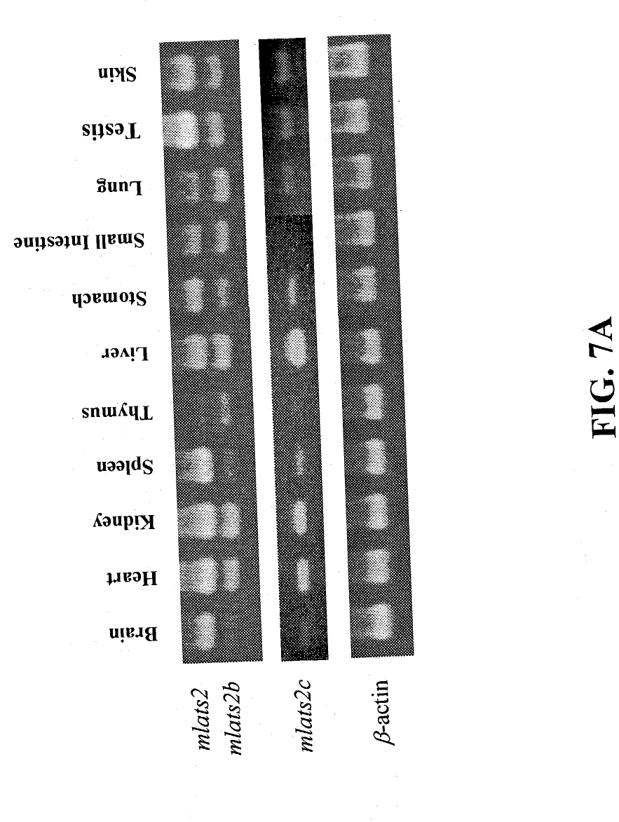

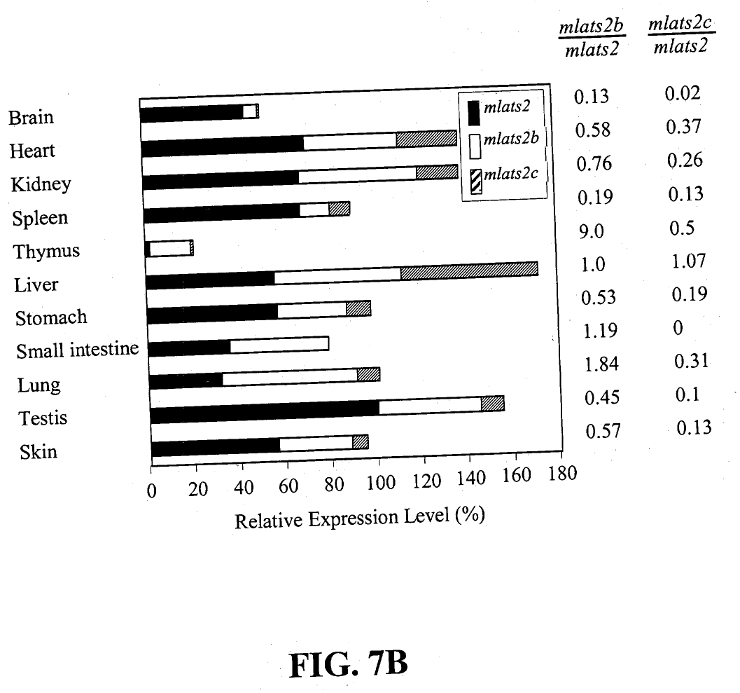

Expression of mlats2, mlats2b, and mlats2c in murine bone marrow was confirmed by RT-PCR employing primer sets specific for individual transcripts. The PCR products of expected sizes (483 bp for mlats2, 379 bp for mlats2b, and 525 bp for mlats2c) were obtained, as shown in FIG. 6. All PCR products were sequenced to confirm their identity. The same PCR primer pairs were used to examine the expression of mlats2, mlats2b, and mlats2c in various mouse tissues. As shown in FIG. 7A, mlats2 was expressed in most tissues analyzed with the highest level observed in testis. Conversely, expression in thymus was very low. Similarly, mlats2b was also expressed widely. However, the ratios of the expression levels between mlats2 and mlats2b appear to be tissue-specific. In particular, in brain, spleen and testis, expression of mlats2 was much higher than that of mlats2b, as shown in FIG. 7B. In contrast, in thymus and lung, the reversed pattern was observed. Expression of mlats2c was relatively weak in all tissues except liver, in which the expression level of mlats2c was comparable to those of mlats2 and mlats2b.

Thus, evidence is provided for alternative splicing of the mouse LATS2 gene. Using the 3′-RACE technique, two novel cDNA fragments, mlats2b and mlats2c, are identified, encoding shorter versions of mLATS2 with distinct C-termini. Alignment of the nucleotide sequences of these two clones with mlats2, hlats2/kpm, and the corresponding human genomic DNA sequence, shown in FIG. 8, reveals a putative intron at the location where the sequence identity between these two clones and mlats2 breaks off. Furthermore, all three genes were expressed in bone marrow. These results indicate that mlats2b and mlats2c are the products of alternative splicing.

The results of tissue profiling using RT-PCR, shown in FIGS. 7A-B, indicate that mlats2, mlats2b, and mlats2c are expressed in almost all mouse tissues examined. However, the relative expression levels appear to be tissue-specific. Consistent with the previous report (Yabuta et al., “Structure, Expression, and Chromosome Mapping of LATS2, a Mammalian Homologue of the Drosophila Tumor Suppressor Gene Lats/Warts,” Genomics 63(2):263-70 (2000), which is hereby incorporated by reference in its entirety), mlats2 was highly expressed in testis. While expression of mlats2c was low in most tissues analyzed, in the liver, the level of mlats2c was comparable to those of mlats2 and mlats2b, shown in FIG. 7A.

To investigate whether the hLATS2/KPM gene is also alternatively spliced, a BLAST search was performed using the cDNA sequence of hlats2/kmp against the GenBank EST database. An EST clone (GenBank accession number AW955972) was found to have an identical sequence to hlats2/kpm at the 5′-end but a distinct 3′-end. Whether the EST clone results from alternative splicing of the hLATS2/KPM gene remains to be determined. The hypothetical splicing site of this clone is different from the one described herein.

One important function of alternative splicing is to produce a functional variant by including or excluding domains important for protein-protein interaction, transcriptional activation or catalytic activity. In particular, several cell cycle regulators are expressed in different forms as a result of alternative splicing. For example, three splicing variants of the human CDC25B have been identified and shown to exhibit different phosphatase activity in vivo (Baldin et al., “Alternative Splicing of the Human CDC25B Tyrosine Phosphatase. Possible Implications for Growth Control?” Oncogene 14(20):2485-95 (1997), which is hereby incorporated by reference in its entirety). Another example is p10, an alternatively spliced form of the human p15 cyclin-dependent kinase (CDK) inhibitor. In contrast to p15, p10 does not bind to CDK4 or CDK6 (Tsubari et al., “Cloning and Characterization of p10, an Alternatively Spliced Form of p15 Cyclin-Dependent Kinase Inhibitor,” Cancer Research 57(14):2966-73 (1997), which is hereby incorporated by reference in its entirety). In addition, the respective splicing variants of cyclin C, D1 and E, which have distinct expression patterns and functions, have been identified (Li et al., “Alternatively Spliced Cyclin C mRNA is Widely Expressed, Cell Cycle Regulated, and Encodes a Truncated Cyclin Box,” Oncogene 13(4):705-12 (1996); Sawa et al., “Alternatively Spliced Forms of Cyclin D1 Modulate Entry into the Cell Cycle in an Inverse Manner,” Oncogene 16(13):1701-12 (1998); Sewing et al., “Alternative Splicing of Human Cyclin E,” J. Cell Science 107(Pt 2):581-8 (1994); Mumberg et al., “Cyclin ET, a New Splice Variant of Human Cyclin E With a Unique Expression Pattern During Cell Cycle Progression and Differentiation,” Nucleic Acids Res. 25(11):2098-105 (1997), which are hereby incorporated by reference in their entirety). Alternative splicing therefore appears to occur frequently in the genes encoding cell cycle regulators. The results indicate that the LATS2 gene, which has been implicated in cell cycle control, is also subject to regulation through alternative splicing.

### Example 14

Circadian Expression Profiles of mlats2 and mlats2b

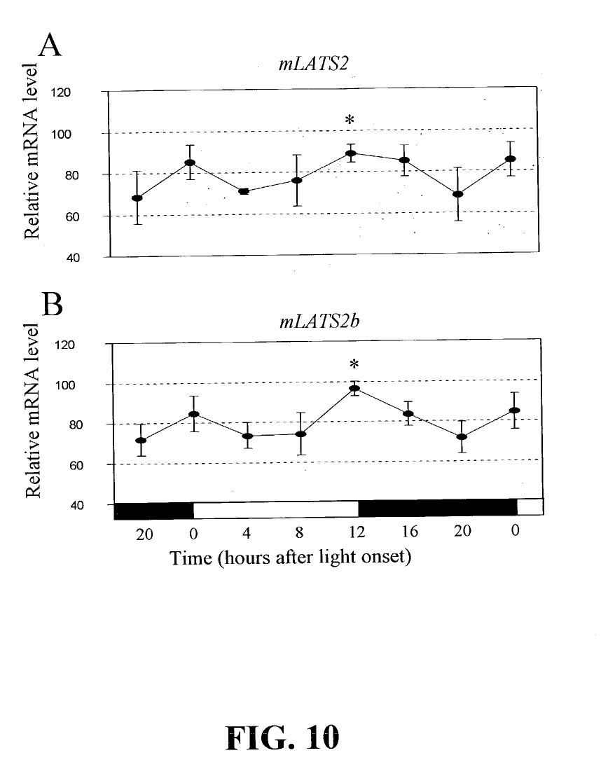

Although the initial relative quantitative RT-PCR result confirmed the circadian expression pattern of clone 6A-2-9 obtained from the RAP-PCR screening, shown in FIGS. 2A-B, the primer set used for the analysis amplified all three transcripts, mlats2, mlats2b, and mlats2c. To further determine the circadian expression profiles of mlats2 and mlats2b, relative quantitative RT-PCR was performed using the primer set specific for mlats2 or mlats2b. As shown in FIGS. 10A-B, the circadian expression profiles of mlats2 (FIG. 10A) and mlats2b (FIG. 10B) were very similar. Both oscillated within 24 hours and peaked at 12 hours after light onset. When the circadian expression patterns of mlats2 and mlats2b were compared to that of clone 6A-2-9, both similarity and discrepancy were observed (FIGS. 2A-B and FIGS. 10A-B). The mean values at 0 and 12 hours after light onset were always higher than those at their preceding and subsequent time points. However, the expression level of clone 6A-2-9 exhibited a peak at time 0. Therefore, it is possible that one or more splice variants remain to be identified. Alternatively, mlats2c could be highly expressed at time 0.

### Example 15

mLATS2 is Functionally Regulated by mLATS2b

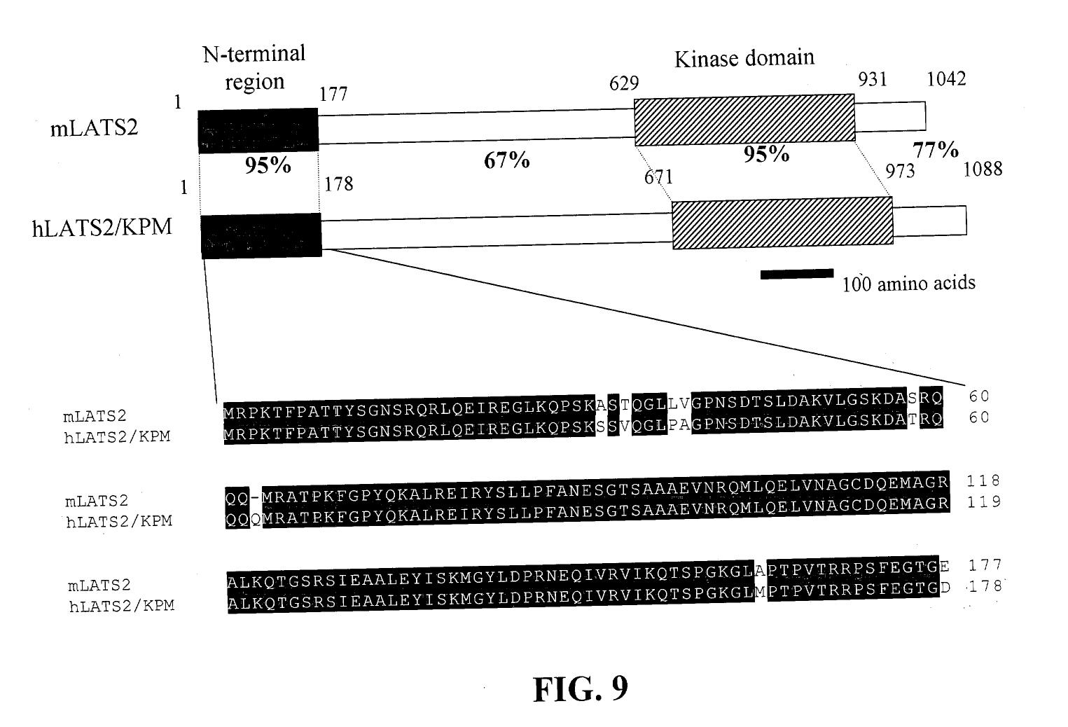

The kinase domain located near the C-terminus of LATS2 is highly conserved between human and mouse proteins. It is noteworthy that the other highly conserved region is the N-terminal domain of LATS2, shown in FIG. 9. It is possible that this region is important for protein-protein interaction. It is therefore interesting that mLATS2b has the same N-terminus as that of mLATS2 while lacking the kinase domain.

It is plausible that the role of mLATS2b is to modulate the function of mLATS2 via competitive binding to a target protein. To elucidate the role of mLATS2b, the protein-interaction partners of mLATS2b were searched using yeast two-hybrid screening. Forty-seven positive clones were obtained after screening more than 10⁶ clones of the human bone marrow cDNA library. These mLATS2b-interacting proteins include proteins involved in translation, cytoskeleton remodeling, signal transduction, and metabolic pathways. One of these proteins, the Replication Protein Binding Trans-Activator (RBT1) previously identified as a transcriptional co-activator associated with Replication Protein A (Cho et al., “RBT1, a Novel Transcriptional Co-Activator, Binds the Second Subunit of Replication Protein A,” Nucleic Acids Res 28(18):3478-85 (2000), which is hereby incorporated by reference in its entirety), is particularly interesting because it may play a role in the regulation of DNA replication.

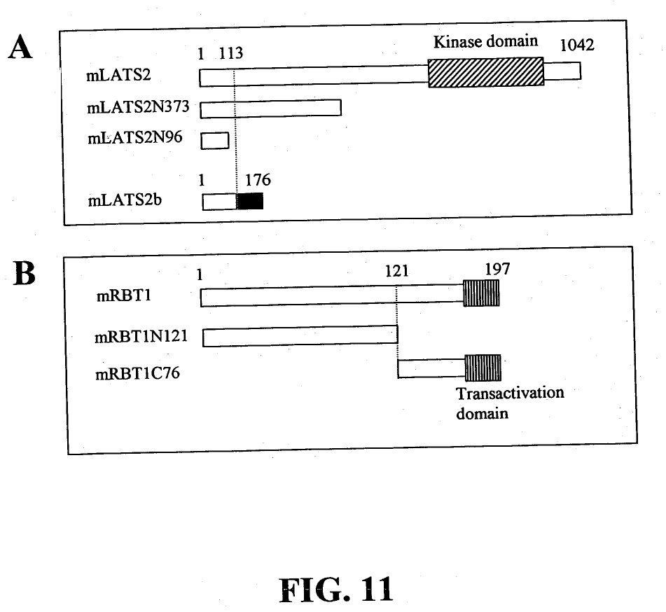

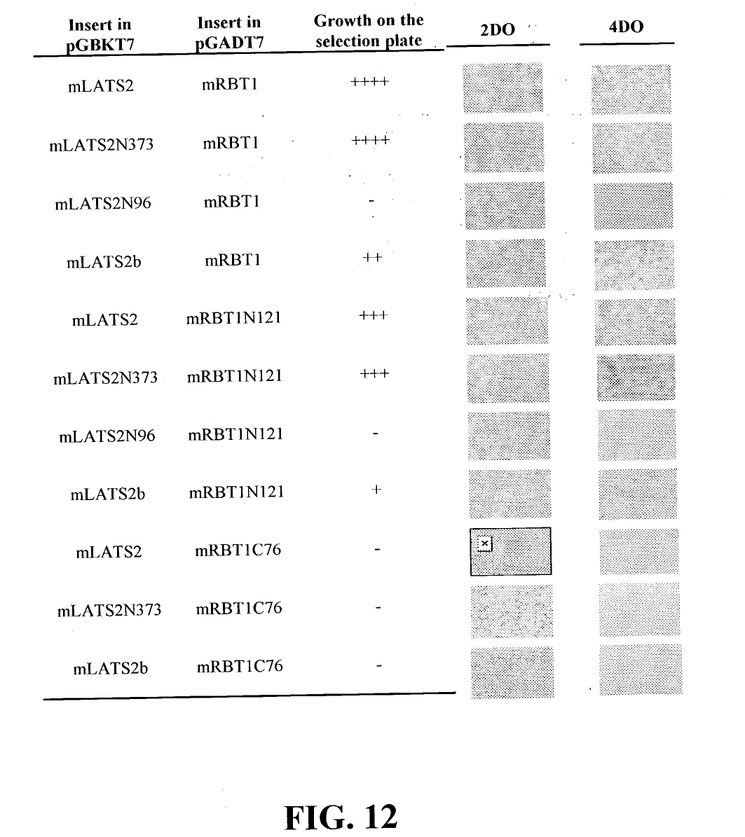

The interaction between mRBT1 and mLATS2/2b was further characterized by the yeast two-hybrid assay. The positions of the inserts used in the assays are depicted in FIGS. 11A-B and the results are summarized in FIG. 12. As expected, mLATS2 also interacted with mRBT1. Since a comparable result was obtained with the N-terminal 373 amino acids of mLATS2 (mLATS2N373), therefore, the kinase domain does not interfere with the interaction between mRBT1 and mLATS2. The N-terminal 96 amino acids of mLATS2/2b (mLATS2N96), however, did not interact with mRBT1. The N-terminal 121 amino acids of mRBT1 (mRBT1N121) could interact with mLATS2, mLATS2N373, and mLATS2b, but not with mLATS2N96. In contrast, the C-terminal 76 amino acids of mRBT1 (mRBT1C76), which contains the transactivation domain, did not interact with mLATS2/2b. Considering the fact that mLATS2 and mLATS2b share the same N-terminal 113 amino acids, the data shown here suggest that the RBT1-interacting region of mLATS2/2b is located between amino acids 96 and 113.

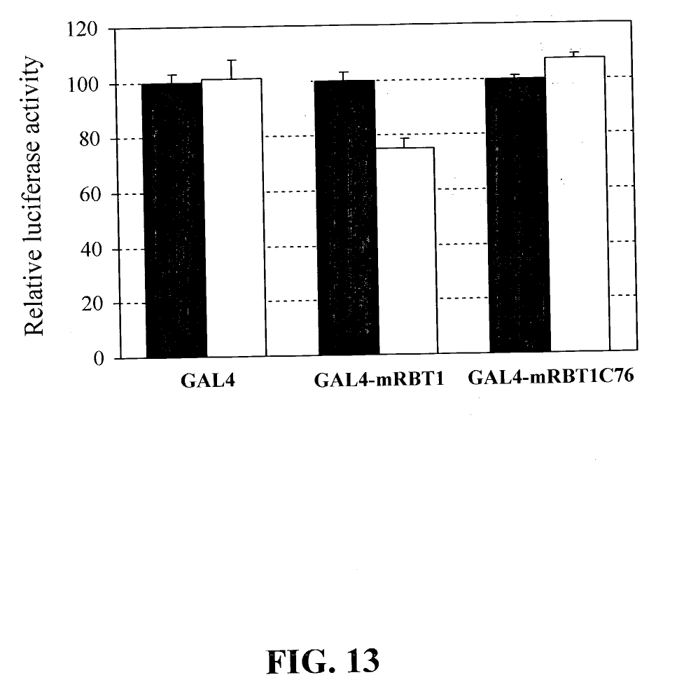

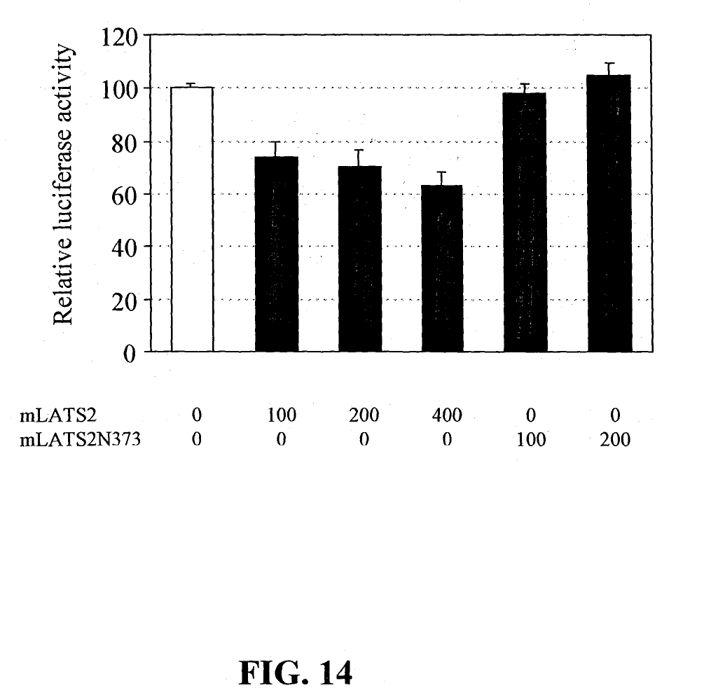

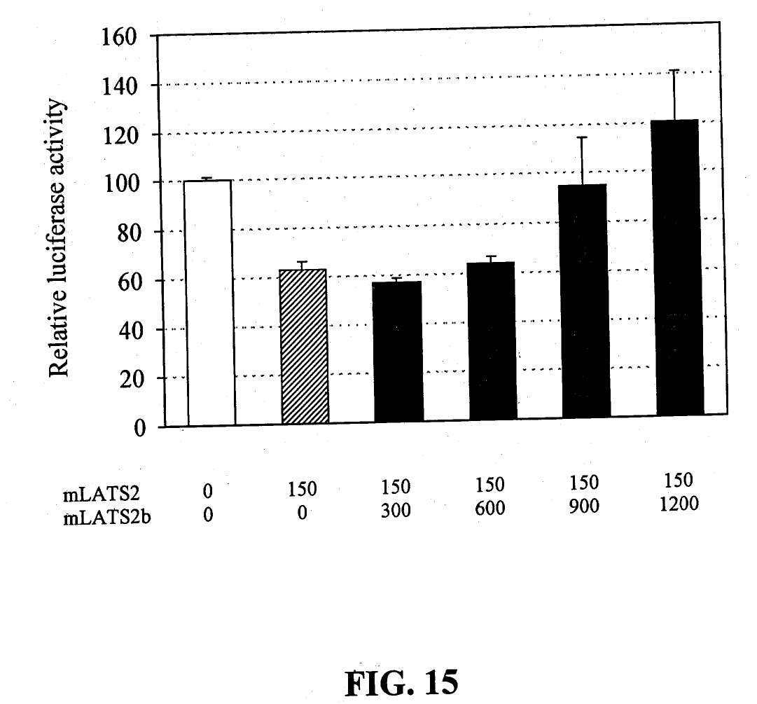

As RBT1 has a transactivation domain located in its C-terminal region (Cho et al., “RBT1, a Novel Transcriptional Co-Activator, Binds the Second Subunit of Replication Protein A,” Nucleic Acids Res 28(18):3478-85 (2000), which is hereby incorporated by reference in its entirety), the effects of mLATS2 and mLATS2b on RBT1 were determined in the content of the mammalian one-hybrid assay. Consistent with the previous report, when fused to the GAL4 DNA binding domain, both full-length and C-terminal 76 amino acids of mRBT1 showed high levels of transcriptional activity (>1000 fold when compared with GAL4 alone) in the content of the mammalian one-hybrid assay. In the presence of mLATS2, the transcriptional activity of mRBT1 was significantly inhibited, as shown in FIG. 13. The inhibitory effect of mLATS2 was specific because the transcriptional activity of the GAL4 DNA-binding domain was not affected by mLATS2, as shown in FIG. 13. Furthermore, the inhibitory effect of mLATS2 on mRBT1 was dependent on their interaction, as the activity of the mRBT1 C-terminal 76 amino acids (mRBT1C76), which did not interact with mLATS2 in the yeast two-hybrid assay, was not negatively regulated by mLATS2 as shown in FIG. 13. Deletion of the kinase domain completely abolished the inhibitory effect of mLATS2 on the transcriptional activity of mRBT1, as shown in FIG. 14. Finally, the inhibitory effect of mLATS2 on mRBT1 transcriptional activity was antagonized by mLATS2b, shown in FIG. 15.

Thus, a cDNA fragment corresponding to the 5′ region of mlats2 was cloned in the murine bone marrow when gene expression patterns at six different time points were compared. The warts/lats gene was first identified in Drosophila as a tumor suppressor gene (Xu et al., “Identifying Tumor Suppressors in Genetic Mosaics: the Drosophila Lats Gene Encodes a Putative Protein Kinase,” Development 121(4):1053-63 (1995), which is hereby incorporated by reference in its entirety). The human and mouse homologues of the warts/lats gene, namely lats1 and lats2, were subsequently identified (Yabuta et al., “Structure, Expression, and Chromosome Mapping of LATS2, a Mammalian Homologue of the Drosophila Tumor Suppressor Gene Lats/Warts,” Genomics 63(2):263-70 (2000); Tao et al., “Human Homologue of the Drosophila Melanogaster Lats Tumour Suppressor Modulates CDC2 Activity,” Nature Genetics 21(2):177-81 (1999); Nishiyama et al., “A Human Homolog of Drosophila Warts Tumor Suppressor, h-warts, Localized to Mitotic Apparatus and specifically Phosphorylated During Mitosis,” FEBS Letters 459(2):159-65 (1999); Hori et al., “Molecular Cloning of a Novel Human Protein Kinase, kpm, That is Homologous to Warts/Lats, a Drosophila Tumor Suppressor,” Oncogene 19:3101-3109 (2000), which are hereby incorporated by reference in their entirety). All LATS proteins contain a serine/threonine kinase domain highly homologous to the catalytic domain of the myotonic dystrophy protein kinase (DMPK) family. The DMPK family proteins such as Dbf2 and Orb6 in yeast and Citron-K kinase in human have been shown to function during the mitotic phase. The kinase activity of Dbf2 is cell-cycle-regulated with its activity peaking in the late mitotic phase Toyn et al., “The Dbf2 and Dbf20 Protein Kinases of Budding Yeast are Activated After the Metaphase to Anaphase Cell Cycle transition,” EMBO J 13(5):1103-13 (1994), which is hereby incorporated by reference in its entirety). For the temperature-sensitive Dbf2 mutant, the cells arrested in telophase with elongated spindles under the non-permissive temperature. Orb6 is required to maintain polarity of the actin cytoskeleton during the interphase and to promote actin reorganization both after mitosis and during the activation of bipolar growth (Verde et al., “Fission Yeast orb6, a ser/thr Protein Kinase Related to Mammalian Rho Kinase and Myotonic Dystrophy kinase, is Required for Maintenance of Cell Polarity and Coordinates Cell Morphogenesis With the Cell Cycle,” Proc Natl Acad Sci USA 95(13):7526-31 (1998), which is hereby incorporated by reference in its entirety). Overexpression of orb6 led to an increase in cell length at division, indicating that onset of mitosis is delayed. Citron-K kinase has been shown to localize to the cleavage furrow of dividing cells and overexpression of citron-K kinase resulted in multinucleated cells (Madaule et al., “Role of Citron kinase as a Target of the Small GTPase Rho in Cytokinesis,” Nature 394(6692):491-4 (1998), which is hereby incorporated by reference in its entirety).

Similarly, evidence indicating the involvement of LATS1 and LATS2 in cell cycle regulation has also evolved. For example, it has been shown that phosphorylation of hLATS1 is cell cycle-dependent and the phosphorylated hLATS1 negatively regulates CDC2 activity by forming the hLATS1-CDC2 complex in the mitotic phase (Tao et al., “Human Homologue of the Drosophila Melanogaster Lats Tumour Suppressor Modulates CDC2 Activity,” Nature Genetics 21(2):177-81 (1999), which is hereby incorporated by reference in its entirety). In addition, hLATS1 has been reported to localize at the centrosome in the interphase and translocate towards mitotic spindles in the metaphase and anaphase (Nishiyama et al., “A Human Homolog of Drosophila Warts Tumor Suppressor, h-warts, Localized to Mitotic Apparatus and Specifically Phosphorylated During Mitosis,” FEBS Letters 459(2):159-65 (1999), which is hereby incorporated by reference in its entirety). High incidence of soft-tissue sarcomas and ovarian stromal cell tumors in the lats1^{-/-} mice also supports the role of LATS1 in cell cycle control (St. John et al., “Mice Deficient of Lats1 Develop Soft-Tissue Sarcomas, Ovarian Tumours and Pituitary Dysfunction,” Nature Genetics 21(2):182-6 (1999), which is hereby incorporated by reference in its entirety). Furthermore, when introduced into lats1-deficient mouse cells; hLATS1 caused cell cycle arrest in the G2/M phase through the inhibition of CDC2 kinase activity (Yang et al., “Human Homologue of Drosophila Lats, LATS1, Negatively Regulate Growth by Inducing G(2)/M Arrest or Apoptosis,” Oncogene 20(45):6516-23 (2001), which is hereby incorporated by reference in its entirety). The human KPM protein (identical to hLATS2) has been shown to undergo phosphorylation during the mitotic phase and has been suggested to play a role in the progression of mitosis (Hori et al., “Molecular Cloning of a Novel Human Protein Kinase, kpm, That is Homologous to Warts/Lats, a Drosophila Tumor Suppressor,” Oncogene 19:3101-3109 (2000), which is hereby incorporated by reference in its entirety). Furthermore, expression of hLATS2 is induced by p53, a tumor suppressor gene involved in cell cycle control (Kostic et al., “Isolation and Characterization of Sixteen Novel p53 Response Genes,” Oncogene 19(35):3978-87 (2000), which is hereby incorporated by reference in its entirety).

In the present invention two splice variants, mlats2b and mlats2c, are disclosed as encoding shorter versions of mLATS2. One important function of alternative splicing is to produce a functional variant by including or excluding domains important for protein-protein interaction, transcriptional activation or catalytic activity. In particular, several cell cycle regulators are expressed in different forms as a result of alternative splicing. For example, three splice variants of the human CDC25B have been identified and shown to exhibit different phosphatase activity in vivo (Baldin et al., “Alternative Splicing of the Human CDC25B Tyrosine Phosphatase. Possible Implications for Growth Control?” Oncogene 14(20):2485-95 (1997), which is hereby incorporated by reference in its entirety). Another example is p10, an alternatively spliced form of the human p15 cyclin-dependent kinase (CDK) inhibitor. In contrast to p15, p10 does not bind to CDK4 or CDK6 (Tsubari et al., “Cloning and Characterization of p10, an Alternatively Spliced Form of p15 Cyclin-Dependent Kinase Inhibitor,” Cancer Research 57(14):2966-73 (1997), which is hereby incorporated by reference in its entirety). In addition, the respective splice variants of cyclin C, D1, and E, which have distinct expression patterns and functions, have been reported (Li et al., “Alternatively Spliced Cyclin C mRNA is Widely Expressed, Cell Cycle Regulated, and Encodes a Truncated Cyclin Box,” Oncogene 13(4):705-12 (1996); Sawa et al., “Alternatively Spliced Forms of Cyclin D1 Modulate Entry into the Cell Cycle in an Inverse Manner,” Oncogene 16(13):1701-12 (1998); Sewing et al., “Alternative Splicing of Human Cyclin E,” Journal of Cell Science 107(Pt 2):581-8 (1994); Mumberg et al., “Cyclin ET, a New Splice Variant of Human Cyclin E With a Unique Expression Pattern During Cell Cycle Progression and Differentiation,” Nucleic Acids Research 25(11):2098-105 (1997), which are hereby incorporated by reference in their entirety). Comparison between mLATS2 and mLATS2b revealed that they have the same N-terminal 113 amino acids, as shown in FIG. 3. In addition, the kinase domain is missing in mLATS2b, which strongly suggests that mLATS2b could regulate the function of mLATS2 by competitively binding to the same proteins. This hypothesis was addressed by the identification of proteins that interact with mLATS2/2b. The yeast two-hybrid assays revealed that mRBT1 can interact with both mLATS2 and mLATS2b. In addition, mLATS2 inhibited the transcriptional activity of mRBT1 in the content of the mammalian one-hybrid assay and the inhibitory effect of mLATS2 was antagonized by mLATS2b. Collectively, these data demonstrated that mLATS2b is a negative regulator of mLATS2.

The fact that mLATS2 can negatively regulate mRBT1 further supports a role of mLATS2 as a cell cycle regulator. As a replication protein A (RPA)-interacting protein, it is possible that RBT1 may exert its activity to promote cell proliferation. Indeed, the expression levels of hRBT1 are higher in cancerous cells in comparison to non-transformed cells (Cho et al., “RBT1, a Novel Transcriptional Co-Activator, Binds the Second Subunit of Replication Protein A,” Nucleic Acids Res 28(18):3478-85 (2000), which is hereby incorporated by reference in its entirety). In addition, transactivation of RBT1 was significantly down-regulated by p53 (Cho et al., “RBT1, a Novel Transcriptional Co-Activator, Binds the Second Subunit of Replication Protein A,” Nucleic Acids Res 28(18):3478-85 (2000), which is hereby incorporated by reference in its entirety) although it remains to be determined whether p53 acts through LATS2 to inhibit RBT1.

In conclusion, mlats2 has been identified as a potential clock-controlled gene in murine bone marrow. In addition, it is demonstrated that mLATS2 is negatively regulated by mLATS2b, an mLATS2 isoform generated by alternative splicing. Since circadian variations in the cell cycle status of bone marrow cells have been well documented, the potential role of mLATS2 being a cell cycle regulator signifies the need for further studies of its function and regulation.

Furthermore, because LATS has been identified as a tumor suppressor gene, the present invention provides methods for the detection of disorders of cellular overproliferation, including cancer and hyperproliferative disorders, as indicated by LATS2b or 2c expression, and for methods of diagnosing and treating cancer and hyperproliferative disorders using compositions based on LATS2b or 2c proteins, nucleic acid molecules, iRNA, and anti-LATS2b or 2c antibodies.

In addition since LATS2, 2b, and 2c were found to be expressed in bone marrow, the present invention also provides methods for detecting and treating blood disorders, including leukemia, using the compositions disclosed above. The compositions taught above can also be used to modulate growth and differentiation of hematopoietic cells including stem cells in vivo or in vitro. Because these genes are expressed in many other tissues and organs, the compositions can also be used to modulate growth and differentiation of ells, including stem cells, in these tissues and organs in vivo or in vitro.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. An isolated nucleic acid molecule encoding a LATS2b protein or polypeptide, wherein the nucleic acid molecule either: 1) has a nucleotide sequence of SEQ ID NO: 1; 2) encodes a protein or polypeptide having an amino acid sequence of SEQ ID NO: 2; 3) has a nucleotide sequence that is at least 55% similar to the nucleotide sequence of SEQ ID NO: 1 by basic BLAST analysis; or 4) has a nucleotide sequence that hybridizes to the nucleotide sequence of SEQ ID NO: 1 under stringent conditions characterized by a hybridization buffer comprising 5×SSC buffer at a temperature of 45° C.

2. The isolated nucleic acid molecule according to claim 1, wherein the nucleic acid molecule has a nucleotide sequence of SEQ ID NO: 1.

3. The isolated nucleic acid molecule according to claim 1, wherein the nucleic acid molecule encodes a protein or polypeptide having an amino acid sequence of SEQ ID NO: 2.

4. The isolated nucleic acid molecule according to claim 1, wherein the nucleic acid molecule has a nucleotide sequence that is at least 55% similar to the nucleotide sequence of SEQ ID NO: 1 by basic BLAST analysis.

5. The isolated nucleic acid molecule according to claim 1, wherein the nucleic acid molecule has a nucleotide sequence that hybridizes to the nucleotide sequence of SEQ ID NO: 1 under stringent conditions characterized by a hybridization buffer comprising 5×SSC buffer at a temperature of 45° C.

6. An expression vector comprising a transcriptional and translational regulatory DNA operably linked to the nucleic acid molecule according to claim 1.

7. The expression vector according to claim 6, wherein the nucleic acid molecule is in proper sense orientation and correct reading frame.

8. A host cell transduced with the nucleic acid molecule according to claim 1.

9. The host cell according to claim 8, wherein the host cell is selected from the group consisting of a bacterial cell, a virus, a yeast cell, an insect cell, a fungal cell, and a mammalian cell.

10. The mammalian cell according to claim 9, wherein the nucleic acid molecule either 1) has a nucleotide sequence of SEQ ID NO: 1; 2) ) encodes an amino acid having SEQ ID NO: 2; 3) is at least 55% similar to the nucleotide sequence of SEQ ID NO: 1 by basic BLAST analysis; or 4) hybridizes to the nucleotide sequence of SEQ ID NO: 1 under stringent conditions characterized by a hybridization buffer comprising 5×SSC buffer at a temperature of 45° C.

11. An antisense nucleic acid molecule which comprises a region the same as the nucleic acid molecule according to claim 1 and another region complementary to.

12. An expression vector comprising a transcriptional and translational regulatory DNA operably linked to the antisense nucleic acid molecule according to claim 11.

13. A host cell transformed with the antisense nucleic acid molecule according to claim 11.

14. The host cell according to claim 13, wherein the cell is selected from the group consisting of a bacterial cell, a virus, a yeast cell, an insect cell, a fungal cell, and a mammalian cell.

15. An RNAi nucleic acid molecule which comprises a region the same as and a region complementary to the nucleic acid molecule according to claim 1.

16. An expression vector comprising a transcriptional and translational regulatory DNA operably linked to the RNAi nucleic acid molecule according to claim 15.

17. A host cell transformed with the RNAi nucleic acid molecule according to claim 15.

18. The host cell according to claim 17, wherein the cell is selected from the group consisting of a bacterial cell, a virus, a yeast cell, an insect cell, a fungal cell, and a mammalian cell.

19. An isolated nucleic acid molecule encoding a LATS2c protein or polypeptide, wherein the nucleic acid molecule either: 1) has a nucleotide sequence of SEQ ID NO: 3; 2) encodes a protein or polypeptide having an amino acid sequence of SEQ ID NO: 4; 3) has a nucleotide sequence that is at least 55% similar to the nucleotide sequence of SEQ ID NO: 3 by basic BLAST analysis; or 4) has a nucleotide sequence that hybridizes to the nucleotide sequence of SEQ ID NO: 3 under stringent conditions characterized by a hybridization buffer comprising 5×SSC buffer at a temperature of 45° C.

20. The isolated nucleic acid molecule according to claim 19, wherein the nucleic acid molecule has a nucleotide sequence of SEQ ID NO: 3.

21. The isolated nucleic acid molecule according to claim 19, wherein the nucleic acid molecule encodes a protein or polypeptide having an amino acid sequence of SEQ ID NO: 4.

22. The isolated nucleic acid molecule according to claim 19, wherein the nucleic acid has a nucleotide sequence that is at least 55% similar to the nucleotide sequence of SEQ ID NO: 3 by basic BLAST analysis.

23. The isolated nucleic acid molecule according to claim 19, wherein the nucleic acid molecule has a nucleotide sequence that hybridizes to the nucleotide sequence of SEQ ID NO: 3 under stringent conditions characterized by a hybridization buffer comprising 5×SSC buffer at a temperature of 45° C.

24. An expression vector comprising a transcriptional and translational regulatory DNA operably linked to a nucleic acid molecule according to claim 19.

25. The expression vector according to claim 24, wherein the nucleic acid molecule is in proper sense orientation and correct reading frame.

26. A host cell transformed with nucleic acid molecule according to claim 19.

27. The host cell according to claim 26, wherein the cell is selected from the group consisting of a bacterial cell, a virus, a yeast cell, an insect cell, a fungal cell, and a mammalian cell.

28. The mammalian cell according to claim 27, wherein the nucleic acid molecule either 1) has a nucleotide sequence of SEQ ID NO: 3; 2) encodes an amino acid having SEQ ID NO: 4; 3) is at least 55% similar to the nucleotide sequence of SEQ ID NO: 3 by basic BLAST using default parameters analysis; or 4) hybridizes to the nucleotide sequence of SEQ ID NO: 3 under stringent conditions characterized by a hybridization buffer comprising 5×SSC buffer at a temperature of 45° C.

29. An antisense nucleic acid molecule which is complementary to the nucleic acid molecule according to claim 19.

30. An expression vector comprising a transcriptional and translational regulatory DNA operably linked to the antisense nucleic acid molecule according to claim 29.

31. The host cell transformed with the antisense nucleic acid molecule according to claim 29.

32. The host cell according to claim 31, wherein the cell is selected from the group consisting of a bacterial cell, a virus, a yeast cell, an insect cell, a fungal cell, and a mammalian cell.

33. An RNAi nucleic acid molecule which comprises a region the same as and a region complementary to the nucleic acid molecule according to claim 19.

34. An expression vector comprising a transcriptional and translational regulatory DNA operably linked to the RNAi nucleic acid molecule according to claim 33.

35. A host cell transformed with the RNAi nucleic acid molecule according to claim 33.

36. The host cell according to claim 35, wherein the cell is selected from the group consisting of a bacterial cell, a virus, a yeast cell, an insect cell, a fungal cell, and a mammalian cell.

37. An isolated LATS2b protein or polypeptide.

38. The isolated protein or polypeptide of claim 37, wherein the protein or polypeptide has an amino acid sequence of SEQ ID NO: 2.

39. The isolated protein or polypeptide of claim 37, wherein the protein or polypeptide has an N-terminus which binds to a cell-cycle related protein.

40. The isolated protein or polypeptide of claim 39, wherein the cell-cycle related protein is zyxin or Replication Protein Binding Trans-Activator 1.

41. An isolated antibody which recognizes the protein or polypeptide according to claim 37.

42. The isolated antibody according to claim 41, wherein the antibody is a polyclonal antibody.

43. The isolated antibody according to claim 41, wherein the antibody is a monoclonal antibody.

44. An isolated LATS2c protein or polypeptide.

45. The isolated protein or polypeptide of claim 44, wherein the protein or polypeptide has an amino acid sequence of SEQ ID NO: 4.

46. The isolated protein or polypeptide of claim 44, wherein the protein or polypeptide has an N-terminus which binds to a cell-cycle related protein.

47. The isolated protein or polypeptide of claim 46, wherein the cell-cycle related protein is zyxin or Replication Protein Binding Trans-Activator 1.

48. An isolated antibody which recognizes the protein or polypeptide according to claim 44.

49. The isolated antibody according to claim 48, wherein the antibody is a polyclonal antibody.

50. The isolated antibody according to claim 48, wherein the antibody is a monoclonal antibody.

51. A composition comprising
a pharmaceutical carrier; and
an antibody against an antigen, wherein the antigen is the protein or polypeptide according to claim 37.

52. The composition according to claim 51 further comprising a cytotoxic component.

53. A method of detecting the expression of LATS2b in a biological sample comprising:
providing an antibody or binding portion thereof that recognizes the LATS2b polypeptide or protein;
contacting the antibody or binding portion thereof with a biological sample; and
detecting any binding that occurs between biological sample and the antibody or binding portion thereof, thereby detecting the expression of LATS2b in the biological sample.

54. The method according to claim 53 further comprising:
providing a LATS2b standard; and
quantifying the amount of LATS2b present in the biological sample by comparing the standard to binding that occurs between the biological sample and the antibody or binding portion thereof.

55. The method according to claim 53, wherein an antibody is used to carry out the method and the antibody is selected from the group consisting of a monoclonal antibody and a polyclonal antibody.

56. The method according to claim 53, wherein a binding portion thereof is used to carry out the method and the binding portion is selected from the group consisting of an Fab fragment, an F(ab′)₂ fragment, and an Fv fragment.

57. The method according to claim 53, wherein the antibody or binding portion thereof has a label to permit detection of binding of the antibody or binding portion thereof to a biological sample and the label is selected from the group consisting of a fluorescent label, a radioactive label, a biologically-active enzyme label, a nuclear magnetic resonance active label, a luminescent label, and a chromophore label.

58. The method according to claim 53, wherein the detecting step is carried out using an assay selected from the group consisting of a western blot, immunoassay, ELISA assay, flow cytometry, radiography, and immunoscintography.

59. The method according to claim 53, wherein the antibody or binding portion thereof is administered to a subject, and said detecting is carried out using an in vivo detection method.

60. The method according to claim 59, wherein the subject is human.

61. A method of detecting LATS2b expression in a biological sample comprising:
providing a nucleic acid molecule that specifically hybridizes to a gene encoding a LATS2b polypeptide or protein, a probe thereto or primers derived therefrom;
contacting the nucleic acid molecule encoding a LATS2b polypeptide or protein, a probe thereto or primers derived therefrom with the biological sample; and
detecting whether the nucleic acid molecule has undergone any hybridization, thereby detecting LATS2b expression in the biological sample.

62. The method according to claim 61, wherein the nucleic acid molecule is selected from the group consisting of oligonucleotide sequences, complementary DNA and RNA, and peptide nucleic acids.

63. The method according to claim 61 further comprising:
providing a LATS2b standard; and
quantifying the amount of LATS2b present in the biological sample by comparing the standard to the hybridization that occurs between the biological sample and the nucleic acid molecule that specifically hybridizes to a gene encoding a LATS2b polypeptide or protein.

64. The method according to claim 61, wherein the detecting is carried out by Northern blot, Southern blot, PCR, reverse transcriptase PCR, in situ hybridization, or in situ PCR.

65. A method of treating a disease condition in a subject comprising:
providing a therapeutic amount of a pharmaceutical conjugate comprising an antibody against a LATS2b protein or polypeptide and a cytotoxic component; and
administering said conjugate to a subject under conditions effective to form an immune complex with a LATS2b polypeptide or protein, thereby treating a disease condition.

66. The method according to claim 65, wherein the subject is human.

67. The method according to claim 65, wherein said administering is carried out orally, parenterally, subcutaneously, intravenously, intramuscularly, intraperitoneally, by intravesical instillation, by intracavitary, intravesical instillation, intraocularly, intraarterially, intralesionally, or by application to mucous membrane.

68. A composition comprising:
a pharmaceutical carrier; and
an antibody against an antigen, wherein the antigen is the protein or polypeptide according to claim 44.

69. The composition according to claim 68 further comprising a cytotoxic component.

70. A method of detecting the expression of LATS2c in a biological sample comprising:
providing an antibody or binding portion thereof which recognizes the LATS2c polypeptide or protein;
contacting the antibody or binding portion thereof with a biological sample; and
detecting any binding that occurs between the biological sample and the antibody or binding portion thereof, thereby detecting the expression of LATS2c in the biological sample.

71. The method according to claim 70 further comprising:
providing a LATS2c standard; and
quantifying the amount of LATS2c present in the biological sample by comparing the standard to binding that occurs between the biological sample and the antibody or binding portion thereof.

72. The method according to claim 70, wherein an antibody is used to carry out the method and the antibody is selected from the group consisting of a monoclonal antibody and a polyclonal antibody.

73. The method according to claim 70, wherein a binding portion thereof is used to carry out the method and the binding portion is selected from the group consisting of an Fab fragment, an F(ab′)₂ fragment, and an Fv fragment.

74. The method according to claim 70, wherein the antibody or binding portion thereof has a label to permit detection of binding of the antibody or binding portion thereof to a biological sample and the label is selected from the group consisting of a fluorescent label, a radioactive label, a biologically-active enzyme label, a nuclear magnetic resonance active label, a luminescent label, and a chromophore label.

75. The method according to claim 70, wherein the detecting step is carried out using an assay selected from the group consisting of a western blot, immunoassay, ELISA assay, flow cytometry, radiography, and immunoscintography.

76. The method according to claim 70, wherein the antibody or binding portion thereof is administered to a subject, and said detecting is carried out using an in vivo detection method.

77. The method according to claim 77, wherein the subject is human.

78. A method of detecting LATS2c expression in a biological sample comprising:
providing a nucleic acid molecule that specifically hybridizes to a gene encoding a LATS2c polypeptide or protein, a probe thereto or primers derived therefrom;
contacting the nucleic acid molecule encoding a LATS2c polypeptide or protein, a probe thereto or primers derived therefrom with the biological sample; and
detecting whether the nucleic acid molecule has undergone any hybridization, thereby detecting LATS2c expression in the biological sample.

79. The method according to claim 78, wherein the nucleic acid molecule is selected from the group consisting of oligonucleotide sequences, complementary DNA and RNA, and peptide nucleic acids.

80. The method according to claim 78 further comprising:
providing a LATS2c standard; and
quantifying the amount of LATS2c present in the biological sample by comparing the standard to the hybridization that occurs between the biological sample and the nucleic acid molecule that specifically hybridizes to a gene encoding a LATS2c polypeptide or protein.

81. The method according to claim 78, wherein the detecting is carried out by Northern blot, Southern blot, PCR, reverse transcriptase PCR, in situ hybridization, or in situ PCR.

82. A method of treating a disease condition in a subject comprising:
providing a therapeutic amount of a pharmaceutical conjugate comprising an antibody against a LATS2c protein or polypeptide and a cytotoxic component; and
administering the conjugate to a subject under conditions effective to form an immune complex with a LATS2c polypeptide or protein, thereby treating a disease condition in the subject.

83. The method according to claim 82, wherein the subject is human.

84. The method according to claim 82, wherein said administering is carried out orally, parenterally, subcutaneously, intravenously, intramuscularly, intraperitoneally, by intravesical instillation, by intracavitary, intravesical instillation, intraocularly, intraarterially, intralesionally, or by application to mucous membrane.

85. A method of regulating LATS2b expression in a subject comprising:
administering to the subject the antisense nucleic acid according to claim 11, thereby regulating LATS2b expression in the subject.

86. A method of regulating LATS2c expression in a subject comprising;
administering to the subject the antisense nucleic acid according to claim 29, thereby regulating LATS2c expression in the subject.

87. A method of gene therapy comprising:
administering to a subject the nucleic acid molecule according to claim 1 or a fragment thereof or a vector expressing a LATS2b protein, polypeptide or fragment thereof.

88. The method according to claim 87, wherein the subject is a mammal.

89. The method according to claim 87, wherein the mammal is human.

90. A method of gene therapy comprising:
administering to a subject the nucleic acid molecule according to claim 19, a fragment thereof, or a vector expressing LATS2c protein, polypeptide or fragment thereof.

91. The method according to claim 90, wherein the subject is a mammal.

92. The method according to claim 91, wherein the mammal is human.

93. A transgenic animal wherein the animal has an altered expression of LATS2b.

94. A transgenic animal whose somatic and germ cells lack a gene encoding a LATS2b protein or polypeptide, or possess a disruption in that gene, whereby the animal exhibits a lack of expression of LATS2b.

95. A transgenic animal wherein the animal has an altered expression of LATS2c.

96. A transgenic animal whose somatic and germ cells lack a gene encoding a LATS2c protein or polypeptide, or possess a disruption in that gene, whereby the animal exhibits a lack of expression of LATS2c.

97. A method of screening drugs that regulate LATS2b activity comprising:
providing the LATS2b protein or polypeptide according to claim 37;
providing a reagent upon which LATS2b exerts activity;
providing a test compound;
blending the LATS2b protein or polypeptide, the reagent, and the test compound to form a mixture;
determining the activity of LATS2b upon the reagent in the mixture; and
measuring any difference between the activity of LATS2b upon the reagent with and without the test compound.

98. A method of screening for drugs that regulate LATS2c activity comprising:
providing the LATS2c protein or polypeptide according to claim 44;
providing a reagent upon which LATS2c exerts activity;
providing a test compound;
blending the LATS2c protein or polypeptide, the reagent, and the test compound to form a mixture;
determining the activity of LATS2c upon the reagent in the mixture; and
measuring any difference between the activity of LATS2c upon the reagent with and without the test compound.

99. A method of screening for drugs that regulate LATS2b expression comprising:
transforming a host cell with a nucleic acid construct comprising a nucleic acid molecule encoding a LATS2b protein or polypeptide operably linked to transcriptional and translational regulatory elements;
culturing the transformed cells;
adding a test compound to the culture containing the transformed cells; and
determining whether the test compound regulates the expression of LATS2b in the transformed cells.

100. The method according to claim 99, wherein the host cell is selected from the group consisting of a bacterial cell, a virus, a yeast cell, an insect cell, a fungal cell, and a mammalian cell.

101. The method according to claim 100, wherein the host cell is a mammalian cell.

102. The method according to claim 101, wherein the mammalian cell is a human cell.

103. A method of screening for drugs that regulate LATS2b expression comprising:
isolating cells from a transgenic animal having altered expression of LATS2b;
adding a test compound to the isolated cells; and
determining whether the test compound regulates the expression of LATS2b in the isolated cells.

104. A method of screening for drugs that regulate LATS2c expression comprising:
transforming a host cell with a nucleic acid construct comprising a nucleic acid molecule encoding a LATS2c protein or polypeptide operably linked to transcriptional and translational regulatory elements;
culturing the transformed cells;
adding a test compound to the culture containing the transformed cells; and
determining whether the test compound regulates the expression of LATS2c in the transformed cells.

105. The method according to claim 104, wherein the host cell is selected from the group consisting of a bacterial cell, a virus, a yeast cell, an insect cell, a fungal cell, and a mammalian cell.

106. The method according to claim 105, wherein the host cell is a mammalian cell.

107. The method according to claim 106, wherein the host cell is a human cell.

108. A method of screening for drugs that regulate LATS2c expression comprising:
isolating cells from a transgenic animal having altered expression of LATS2c;
adding a test compound to the isolated cells; and
determining whether the test compound regulates the expression of LATS2c in the cells.

109. A method of treating a disease condition in a subject comprising:
providing a nucleic acid molecule encoding a LATS2b protein or polypeptide or probe thereto;
contacting the nucleic acid molecule encoding a LATS2b protein or polypeptide or probe thereto with a cell or tissue sample of said subject under conditions effective to bind to cells overexpressing LATS2b from the cell or tissue sample; and
removing cells or tissues which are selected by the nucleic acid molecule or probe thereto, thereby treating a disease condition in the subject.

110. The method according to the claim 109, wherein the subject is human.

111. A method of treating a disease condition in a subject comprising:
providing a labeled antibody or binding protein thereof that recognizes the LATS2b protein or polypeptide or a fragment thereof;
contacting the antibody or binding protein thereof that recognizes the LATS2b protein or polypeptide or a fragment thereof with a cell or tissue sample of said subject under conditions effective to bind to cells overexpressing LATS2b from the cell or tissue sample; and
removing cells or tissues which are selected by the antibody or binding protein thereof, thereby treating a disease condition in the subject.

112. A method of treating a disease condition in a subject comprising:
providing a nucleic acid molecule encoding a LATS2c protein or polypeptide or probe thereto;
contacting the nucleic acid molecule encoding a LATS2c protein or polypeptide or probe thereto with a cell or tissue sample of said subject under conditions effective to bind to cells overexpressing LATS2c from the cell or tissue sample; and
removing cells or tissues which are selected by the nucleic acid molecule or probe thereto, thereby treating a disease condition in the subject.

113. A method according to the claim 112, wherein the subject is human.

114. A method of treating a disease condition in a subject comprising:
providing a labeled antibody or binding protein thereof that recognizes the LATS2c protein or polypeptide or a fragment thereof;
contacting the antibody or binding protein thereof that recognizes the LATS2c protein or polypeptide or a fragment thereof with a cell or tissue sample of said subject under conditions effective to bind to cells overexpressing LATS2c from the cell or tissue sample; and
removing cells or tissues which are selected by the antibody or binding protein thereof, thereby treating a disease condition in the subject.

115. A vaccine comprising:
an antigen comprising a LATS2b protein or polypeptide or antigenic fragment thereof; and
a carrier.

116. A method of treating a disease condition in a subject comprising:
administering a vaccine according to claim 115 to a subject.

117. A method according to claim 116, wherein the subject is human.

118. A vaccine comprising:
an antigen comprising a LATS2c protein or polypeptide or antigenic fragment thereof; and
a carrier.

119. A method of treating a disease condition in a subject comprising:
administering a vaccine according to claim 118 to a subject.

120. A method according to claim 119, wherein the subject is human.

121. A method of regulating cell growth or differentiation comprising:
introducing to cells a vector expressing a LATS2b nucleic acid molecule, thereby regulating the growth or differentiation of the cells.

122. The method according to claim 121, wherein expressing a LATS2b nucleic acid molecule results in down-regulating cell growth or differentiation.

123. The method according to claim 122, wherein the vector comprises a LATS2b nucleic acid molecule inserted in a sense orientation capable of expressing a LATS2b protein or polypeptide.

124. The method according to claim 121, wherein expressing a LATS2b nucleic acid molecule results in up-regulating cell growth or differentiation.

125. The method according to claim 124, wherein the vector comprises an antisense LATS2b nucleic acid molecule.

126. The method according to claim 124, wherein the vector comprises a LATS2b nucleic acid molecule capable of triggering RNAi when expressed.

127. The method according to claim 121, wherein the cells are hematopoietic cells.

128. The method according to claim 121, wherein the cells are stem cells.

129. The method according to claim 121, wherein said introducing is carried out in vivo.

130. The method according to claim 121, wherein said introducing is carried out in vitro.

131. A method of regulating cell growth or differentiation comprising:
introducing to cells a vector expressing a LATS2c nucleic acid molecule, thereby regulating the growth or differentiation of the cells.

132. The method according to claim 131, wherein expressing a LATS2c nucleic acid molecule results in down-regulating cell growth or differentiation.

133. The method according to claim 132, wherein the vector comprises a LATS2c nucleic acid molecule inserted in a sense orientation capable of expressing a LATS2c protein or polypeptide.

134. The method according to claim 131, wherein expressing a LATS2c nucleic acid molecule results in up-regulating cell growth or differentiation.

135. The method according to claim 134, wherein the vector comprises an antisense LATS2c nucleic acid molecule.

136. The method according to claim 134, wherein the vector comprises a LATS2c nucleic acid molecule capable of triggering RNAi when expressed.

137. The method according to claim 131, wherein the cells are hematopoietic cells.

138. The method according to claim 131, wherein the cells are stem cells.

139. The method according to claim 131 wherein said introducing is carried out in vivo.

140. The method according to claim 131, wherein said introducing is carried out in vitro.

141. A method of altering the expression of LATS2 in a cell or subject comprising:
treating a cell with a chemical or molecule capable of interfering with circadian control of the cell, thereby altering the expression of LATS2 in the cell or subject.

142. The method according to claim 141, wherein the chemical is dexamethasone or phorbol-12-myristate-13-acetate.

143. A method of altering the expression of LATS2b in a cell or subject comprising:
treating a cell with a chemical or molecule capable of interfering with circadian control of the cell, thereby altering the expression of LATS2b in the cell or subject.

144. The method according to claim 143, wherein the chemical is dexamethasone or phorbol-12-myristate-13-acetate.

145. A method of altering the expression of LATS2c in a cell or subject comprising:
treating a cell with a chemical or molecule capable of interfering with circadian control of the cell, thereby altering the expression of LATS2c in the cell or subject.

146. The method according to claim 145, wherein the chemical is dexamethasone or phorbol-12-myristate-13-acetate.
